(19) United States
(12) Patent Application Publication
(10) Pub. No.: US 2019/0119298 A1
(43) Pub. Date: Apr. 25, 2019

(54) FUSED 3-PHENYLTETRAMIC ACID DERIVATIVES HAVING HERBICIDAL ACTION

(71) Applicant: Bayer CropScience Aktiengesellschaft, Monheim am Rhein (DE)

(72) Inventors: Franz FENKL, Moembris (DE); Hendrik HELMKE, Liederbach (DE); Andreas REMBIAK, Bad Soden (DE); Alfred ANGERMANN, Kriftel (DE); Stefan LEHR, Liederbach (DE); Reiner FISCHER, Monheim (DE); Guido BOJACK, Wiesbaden-Naurod (DE); Hansjoerg DIETRICH, Liederbach am Taunus (DE); Elmar GATZWEILER, Bad Nauheim (DE); Christopher Hugh ROSINGER, Hofheim (DE)

(21) Appl. No.: 16/093,018

(22) PCT Filed: Apr. 6, 2017
(86) PCT No.: PCT/EP2017/058189

§ 371 Date: Oct. 11, 2018

(30) Foreign Application Priority Data
Apr. 14, 2016 (EP) 16165451.2

(51) Int. Cl. C07D 498/04; A01N 43/72

(52) U.S. Cl. CPC C07D 498/04; A01N 25/32; A01N 43/72

(57) ABSTRACT

The present invention relates to novel herbicidally effective fused 3-phenyltetramic acid derivatives according to the general formula (I) or agrochemically acceptable salts thereof,

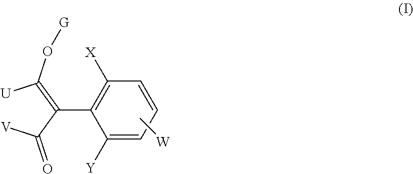

and to the use of these compounds for controlling weeds and weed grasses in crops of useful plants.

The present invention relates to the technical field of crop protection compositions, particularly to that of herbicides for selective control of broad-leaved weeds and weed grasses in crops of useful plants and in the ornamental garden sector and for general control of broad-leaved weeds and weed grasses in areas of the environment where plant growth is disruptive.

The present invention provides novel 3-phenyltetramic acid derivatives, fused to a seven-membered ring, of the general formula (I) or an agrochemically acceptable salt thereof,

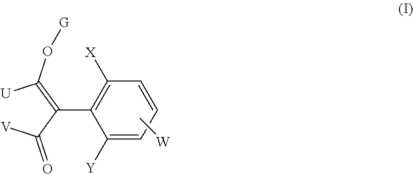

and also a process for their preparation and their use as herbicidal agents for controlling broad-leaved weeds and weed grasses in crops of useful plants.

### BACKGROUND

It is known that certain 3-phenyltetramic acid compounds have herbicidal, insecticidal or fungicidal properties disclosed, for example, in WO 2001/74770, WO 2006056281, WO 2006056282, WO 2005048710, WO 2005044791, DE 19603332, DE 19935963, U.S. Pat. No. 5,811,374, WO 96/35664, WO 99/43649 or WO 2010/102758.

Furthermore, fused 4-phenylpyrazolines are also described, for example, in WO 99/47525 (pinoxaden).

However, the compounds described in the prior art frequently have insufficient herbicidal activity and/or insufficient selectivity in crops of useful plants.

Accordingly, it is an object of the present invention to provide novel compounds which do not have the stated disadvantages.

### DETAILED DESCRIPTION

This object is achieved by novel 3-phenyltetramic acid derivatives, fused to a seven-membered ring, of the general formula (I)

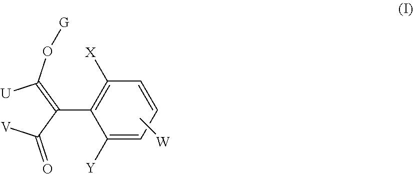

or the agrochemically acceptable salts thereof in which
X represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy or halogen;
Y represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy or halogen;
W represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy, C₂-C₆-alkenyl, C₂-C₆-alkynyl, halogen or phenyl which is unsubstituted or may optionally be substituted by one or more substituents independently of one another selected from the group consisting of C₁-C₃-alkyl, C₁-C₄-haloalkyl, C₃-C₆-cycloalkyl, hydroxy, C₁-C₆-alkoxy, C₁-C₆-haloalkoxy and halogen;
U and V in each case together form a seven-membered ring of the T¹-T⁴ type,

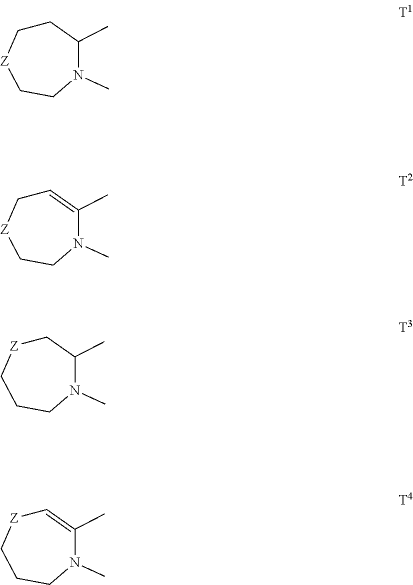

where Z represents an oxygen atom, a group —S(O)ₐ— or a group —N(OR¹)— and
n represents 0, 1 or 2;
represents hydrogen, C₁-C₄-alkyl, C₁-C₄-haloalkyl or C₁-C₄-alkanoyl;
G represents hydrogen, a removable group L or a cation E, where L represents one of the radicals below

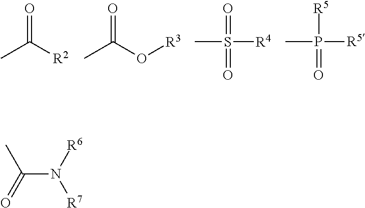

R² represents C₁-C₄-alkyl or C₁-C₃-alkoxy-C₁-C₄-alkyl;
R³ represents C₁-C₄-alkyl;
R⁴ represents C₁-C₄-alkyl or phenyl which is unsubstituted or may optionally be substituted by one or more substituents independently of one another selected from the group consisting of halogen, C₁-C₄-alkyl, C₁-C₄-haloalkyl, C₁-C₄-alkoxy, C₁-C₄-haloalkoxy, nitro or cyano;
R⁵ and R^{5′} each independently of one another represent methoxy or ethoxy;
R⁶ and R⁷ each independently of one another represent methyl, ethyl or phenyl or together form a saturated 5-, 6- or 7-membered ring or together form a saturated 5-, 6- or 7-membered heterocycle having an oxygen or sulfur atom;
E represents an alkali metal ion, an ion equivalent of an alkaline earth metal, an ion equivalent of aluminium, an ion equivalent of a transition metal, a magnesium halogen cation or an ammonium ion, in which optionally one, two, three or all four hydrogen atoms are replaced by identical or different radicals from the groups C₁-C₅-alkyl, C₁-C₆-alkoxy or C₃-C₇-cycloalkyl, which may in each case be substituted one or more times with fluorine, chlorine, bromine, cyano, hydroxy or be interrupted by one or more oxygen or sulfur atoms, or
a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO) or 1,5-diazabicyclo[4.3.0]undec-7-ene (DBU), or a heterocyclic ammonium cation, for example in each case protonated pyridine, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, pyrrole, imidazole, quinoline, quinoxaline, 1,2-dimethylimidazole, 1,3-dimethylimidazolium methyl sulfate, or a sulfonium ion.

The compounds according to the invention are defined in general terms by the formula (I). Preferred substituents or ranges of the radicals given in the formulae mentioned above and below are illustrated hereinafter. The other substituents of the general formula (I) which are not specified hereinafter have the definition given above.

A first embodiment of the present invention encompasses compounds of the general formula (I) in which
X preferably represents hydrogen, C₁-C₄-alkyl, C₁-C₄-haloalkyl, C₁-C₆-alkoxy, C₁-C₃-alkoxy-C₁-C₃-haloalkoxy or halogen, and in which
X particularly preferably represents hydrogen, C₁-C₄-alkyl, methoxy, ethoxy or halogen.

A second embodiment of the present invention encompasses compounds of the general formula (I) in which
Y preferably represents C₁-C₄-alkyl, C₁-C₆-alkoxy, C₁-C₃-alkoxy-C₁-C₄-alkyl, C₁-C₃-haloalkoxy or halogen, in which
Y particularly preferably represents C₁-C₄-alkyl, methoxy, ethoxy or halogen.

A third embodiment of the present invention encompasses compounds of the general formula (I) in which
W preferably represents hydrogen, C₁-C₄-alkyl, C₁-C₄-haloalkyl, C₃-C₆-cycloalkyl, C₁-C₆-alkoxy, C₁-C₆-haloalkoxy, C₂-C₆-alkenyl, C₂-C₆-alkynyl, halogen or phenyl which is unsubstituted or may optionally be substituted by one or more substituents independently of one another selected from the group consisting of C₁-C₃-alkyl, C₁-C₄-haloalkyl, C₃-C₆-cycloalkyl, C₁-C₆-alkoxy, C₁-C₆-haloalkoxy and halogen; and in which
W particularly preferably represents hydrogen, C₁-C₄-alkyl, C₁-C₃-alkoxy, methoxy-C₁-C₂-alkyl, C₁-C₄-haloalkoxy, C₂-C₃-alkenyl, C₂-C₆-alkynyl, halogen or phenyl which is unsubstituted or may optionally be substituted by one or more substituents independently of one another selected from the group consisting of methyl, trifluoromethyl, methoxy, ethoxy, trifluoromethyl and also fluorine, chlorine or bromine.

A fourth embodiment of the present invention encompasses compounds of the general formula (I) in which
U and V preferably in each case together form a seven-membered ring of the T¹-T⁴ type,

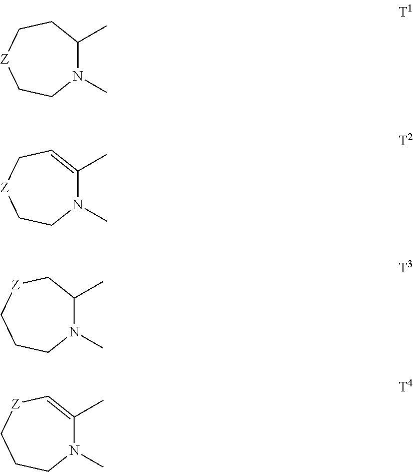

where Z represents an oxygen atom, a group —S(O)ₙ— or a group —N(OR¹)—,
n represents 0, 1 or 2, and
represents C₁-C₄-alkyl, C₁-C₄-haloalkyl or C₁-C₄-alkanoyl; and in which

U and V particularly preferably in each case together form a seven-membered ring of the T¹ or T³ type,

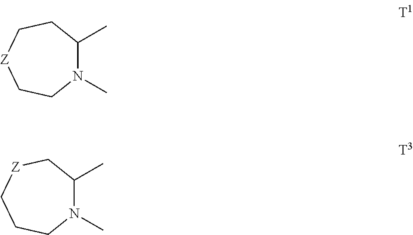

where Z represents an oxygen atom, a group —S(O)ₙ—or a group —N(OCH₃) and
n represents 0, 1 or 2.

A fifth embodiment of the present invention encompasses compounds of the general formula (I) in which
G preferably represents hydrogen, a removable group L or a cation E, where L represents one of the radicals below

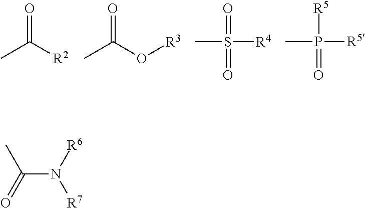

R² represents C₁-C₄-alkyl or C₁-C₃-alkoxy-C₁-C₄-alkyl,
R³ represents C₁-C₄-alkyl,
R⁴ represents C₁-C₄-alkyl or phenyl which is unsubstituted or may optionally be substituted by one or more substituents independently of one another selected from the group consisting of halogen and C₁-C₄-alkyl,

R⁵ and R^{5′} represent methoxy or ethoxy,

R⁶ and R⁷ each independently of one another represent methyl, ethyl or phenyl, and
E represents an alkali metal ion, an ion equivalent of an alkaline earth metal, an ion equivalent
of aluminium, an ion equivalent of a transition metal, a magnesium halogen cation or an ammonium ion, in which optionally one, two, three or all four hydrogen atoms are replaced by identical or different radicals from the groups C₁-C₅-alkyl, C₁-C₆-alkoxy or C₃-C₇-cycloalkyl, or
a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO), 1,5-diazabicyclo[4.3.0]undec-7-ene (DBU) or choline; and in which
G particularly preferably represents hydrogen, a removable group L or a cation E, where L represents one of the radicals below

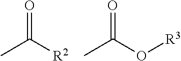

R² represents C₁-C₄-alkyl or C₁-C₃-alkoxy-C₁-C₄-alkyl,
R³ represents C₁-C₄-alkyl, and
E represents an alkali metal ion, an ion equivalent of an alkaline earth metal, an ion equivalent of aluminium, a magnesium halogen cation or an ammonium ion, in which optionally one, two, three or all four hydrogen atoms are substituted by identical or different radicals from the groups C₁-C₅-alkyl, C₁-C₆-alkoxy or C₃-C₇-cycloalkyl, or a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO), 1,5-diazabicyclo[4.3.0]undec-7-ene (DBU) or choline; and in which
G most preferably represents hydrogen, a removable group L or a cation E, where L represents one of the radicals below

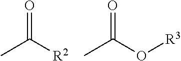

R² represents C₁-C₄-alkyl,
R³ represents methyl or ethyl, and
E represents sodium, potassium, an ion equivalent of calcium, magnesium or aluminium.

In the context of the present invention, it is possible to combine the individual preferred, particularly preferred and most preferred definitions of the substituents X, Y, W, U, V, G, R¹ to R⁷ and E with one another as desired, where the running number n is 0, 1 or 2. This means that the present invention encompasses compounds of the general formula (I) in which, for example, the substituent X has a preferred meaning and the substituents Y and W have the general definition or else the substituent X has a preferred meaning, the substituent Y has a particularly preferred or most preferred meaning and the remaining substituents have a general meaning.

Two of these particularly preferred combinations of the meanings given above for the substituents X, Y, W, U, V, G, R¹ to R⁷ and E are illustrated in an exemplary manner below and each are disclosed as further embodiments:

A sixth embodiment of the present invention encompasses compounds of the general formula (I) in which
X represents hydrogen, C₁-C₄-alkyl, C₁-C₄-haloalkyl, C₁-C₆-alkoxy, C₁-C₃-alkoxy-C₁-C₄-alkyl, C₁-C₃-haloalkoxy or halogen,
Y represents C₁-C₄-alkyl, C₁-C₄-haloalkyl, C₁-C₃-alkoxy-C₁-C₄-alkyl, C₁-C₃-haloalkoxy or halogen,
W represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy, C₂-C₆-alkenyl, C₂-C₆-alkynyl, halogen or phenyl which is unsubstituted or may optionally be substituted by one or more substituents independently of one another selected from the group consisting of C₁-C₃-alkyl, C₁-C₄-haloalkyl, C₃-C₆-cycloalkyl, C₁-C₆-alkoxy, C₁-C₆-haloalkoxy and halogen,
U and V in each case together form a seven-membered ring of the T¹-T⁴ type,

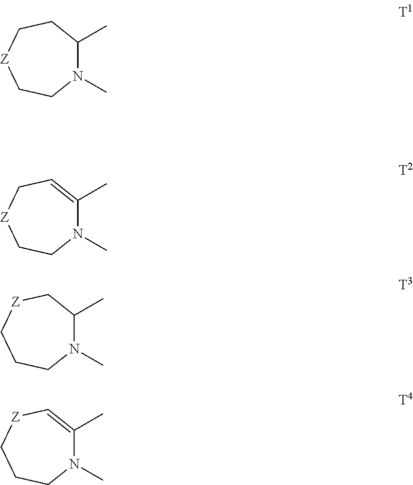

where Z represents an oxygen atom, a group —S(O)₂— or a group —N(OR¹)—,
n represents 0, 1 or 2,
R¹ represents C₁-C₄-alkyl, C₁-C₄-haloalkyl or C₁-C₄-alkanoyl,
G represents hydrogen, a removable group L or a cation E, where L represents one of the radicals below:

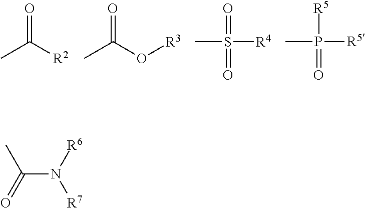

R² represents C₁-C₄-alkyl or C₁-C₃-alkoxy-C₁-C₄-alkyl,
R³ represents C₁-C₄-alkyl,
R⁴ represents C₁-C₄-alkyl or phenyl which is unsubstituted or may optionally be substituted by one or more substituents independently of one another selected from the group consisting of halogen and C₁-C₄-alkyl,
R⁵ and R^{5′} represent methoxy or ethoxy,
R⁶ and R⁷ each independently of one another represent methyl, ethyl or phenyl,
E represents an alkali metal ion, an ion equivalent of an alkaline earth metal, an ion equivalent of aluminium, an ion equivalent of a transition metal, a magnesium halogen cation or an ammonium ion, in which optionally one, two, three or all four hydrogen atoms are replaced by identical or different radicals from the groups C₁-C₅-alkyl, C₁-C₆-alkoxy or C₃-C₇-cycloalkyl, or a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO), 1,5-diazabicyclo[4.3.0]undec-7-ene (DBU) or choline.

A seventh embodiment of the present invention encompasses compounds of the general formula (I) in which
X represents hydrogen, C₁-C₄-alkyl, methoxy, ethoxy or halogen,
Y represents C₁-C₄-alkyl, methoxy, ethoxy or halogen,
W represents hydrogen, C₁-C₄-alkyl, C₁-C₄-haloalkyl, C₁-C₃-alkoxy, methoxy-C₁-C₂-alkyl, C₁-C₄-haloalkoxy, C₂-C₃-alkenyl, C₂-C₆-alkynyl, halogen or phenyl which is unsubstituted or may optionally be substituted by one or more substituents independently of one another selected from the group consisting of methyl, trifluoromethyl, methoxy, ethoxy, trifluoromethyl and also fluorine, chlorine or bromine,
U and V in each case together form a seven-membered ring of the T¹ or T³ type,

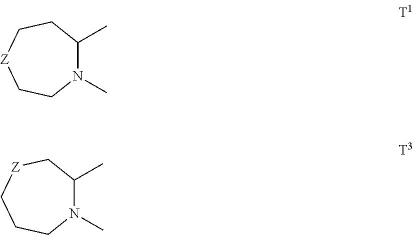

where Z represents an oxygen atom, a group —S(O)ₙ— or a group —N(OCH₃) and
n represents 0, 1 or 2.
G represents hydrogen, a removable group L or a cation E, where L represents one of the radicals below

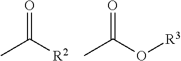

R² represents C₁-C₄-alkyl,
R³ represents methyl or ethyl, and
E represents sodium, potassium, an ion equivalent of calcium, magnesium or aluminium.

In the general formula (I) and in all the formulae below in the present invention, the radicals alkyl, alkoxy, haloalkyl, haloalkoxy, alkylamino and the corresponding unsaturated and/or substituted radicals can in each case be straight-chain or branched in the carbon skeleton. Unless stated specifically, preference is given for these radicals to the lower carbon skeletons, for example those having 1 to 6 carbon atoms, in particular 1 to 4 carbon atoms, or in the case of unsaturated groups having 2 to 6 carbon atoms, in particular 2 to 4 carbon atoms. Alkyl radicals, both alone and in the composite definitions such as alkoxy, haloalkyl, etc., are, for example, methyl, ethyl, n-propyl or isopropyl, n-butyl, isobutyl, tert-butyl or 2-butyl, pentyls, hexyls, such as n-hexyl, isohexyl and 1,3-dimethylbutyl, heptyls, such as n-heptyl, 1-methylhexyl and 1,4-dimethylpentyl; alkenyl and alkynyl radicals have the definition of the possible unsaturated radicals corresponding to the alkyl radicals; where at least one double bond or triple bond is present, preferably one double bond or triple bond, respectively. Alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, ethynyl, propargyl, but-2-yn-1-yl, but-3-yn-1-yl and 1-methylbut-3-yn-1-yl.

Cycloalkyl groups are, for example, cyclopropyl, cyclobutyl, cyclopentyl, cyclohexyl, cycloheptyl and cyclooctyl. The cycloalkyl groups can be present in bi- or tricyclic form.

If haloalkyl groups and haloalkyl radicals of haloalkoxy, haloalkenyl, haloalkynyl etc. are stated, the lower carbon skeletons of these radicals having, for example, 1 to 6 carbon atoms or 2 to 6 carbon atoms, especially 1 to 4 carbon atoms or preferably 2 to 4 carbon atoms, and the corresponding unsaturated and/or substituted radicals are in each case straight-chain or branched in the carbon skeleton. Examples are difluoromethyl, 2,2,2-trifluoroethyl, trifluoroallyl, 1-chloroprop-1-yl-3-yl.

Alkylene groups in these radicals are the lower carbon skeletons, for example those having 1 to 10 carbon atoms, especially 1 to 6 carbon atoms, or preferably 2 to 4 carbon atoms, and also the corresponding unsaturated and/or substituted radicals in the carbon skeleton which may in each case be straight-chain or branched. Examples are methylene, ethylene, n- and isopropylene and n-, s-, iso-, t-butylene.

Hydroxyalkyl groups in these radicals are the lower carbon skeletons, for example those having 1 to 6 carbon atoms, especially 1 to 4 carbon atoms, and also the corresponding unsaturated and/or substituted radicals in the carbon skeleton which may in each case be straight-chain or branched. Examples of these are 1,2-dihydroxyethyl and 3-hydroxypropyl.

Halogen is fluorine, chlorine, bromine or iodine. Haloalkyl, -alkenyl and -alkynyl are alkyl, alkenyl and alkynyl partly or fully substituted by halogen, preferably by fluorine, chlorine or bromine, especially by fluorine and/or chlorine, for example monohaloalkyl, perhaloalkyl, CF₃, CF₂Cl, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same correspondingly applies to haloalkenyl and other halogen-substituted radicals.

Aryl is a monocyclic, bicyclic or polycyclic aromatic system, for example phenyl or naphthyl, preferably phenyl.

The compounds of the formula (I) are capable of forming salts. Salts may be formed by the action of a base on those compounds of the formula (I) that bear an acidic hydrogen atom. Suitable bases are, for example, organic amines such as trialkylamines, morpholine, piperidine or pyridine, and the hydroxides, carbonates and bicarbonates of ammonium, alkali metals or alkaline earth metals, especially sodium hydroxide, potassium hydroxide, sodium carbonate, potassium carbonate, sodium bicarbonate and potassium bicarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NRR′R″R′″]⁺ in which R to R′″ each independently of one another represent an organic radical, in particular alkyl, aryl, aralkyl or alkylaryl. Also suitable are alkylsulfonium and alkylsulfoxonium salts, such as (C₁-C₄)-trialkylsulfonium and (C₁-C₄)-trialkylsulfoxonium salts.

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts will comprise the conjugated base of the acid as the anion.

Suitable substituents present in deprotonated form, such as, for example, sulfonic acids or carboxylic acids, may form inner salts with groups which for their part can be protonated, such as amino groups.

Primarily for reasons of higher herbicidal activity, better selectivity and/or better preparability, compounds of the general formula (I) according to the invention or the agrochemical salts or quaternary N derivatives thereof that are of particular interest are those in which individual radicals have one of the preferred definitions already specified or specified below, or especially those in which one or more of the preferred definitions already specified or specified below occur in combination.

The radical definitions stated above, in general terms or listed within areas of preference, apply both to the end products of the general formula (I) and correspondingly to the starting materials or the intermediates required for their preparation in each case. These radical definitions can be exchanged for one another, i.e. including between the given preferred ranges.

The compounds of the formula (I) can, depending on the type of substituents, be present as geometric and/or optical isomers or isomer mixtures, in differing composition which can optionally be separated in the usual manner Both the pure isomers and also the tautomer, isomer or enantiomer mixtures, their preparation and use, as well as compositions comprising these are provided by the present invention. However, for the sake of simplicity, the terminology used hereinbelow is always compounds of the formula (I) although both the pure compounds and also optionally mixtures with different proportions of isomeric and tautomeric compounds are intended.

Taking the meanings described above for groups T¹-T⁴ into account, the present invention thus comprises the following structure types:

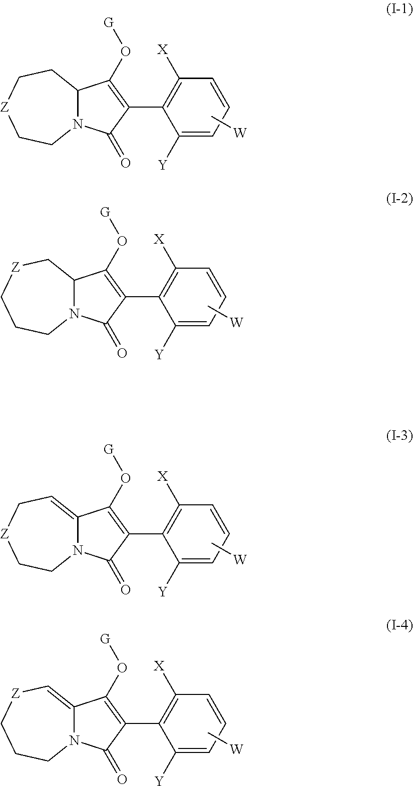

The preparation of the compounds according to the invention of the general formula (I) is carried out analogously to processes known from the literature, for example by

a) if G represents a hydrogen atom, cyclising precursors of the general formulae (II-1 to II-4)

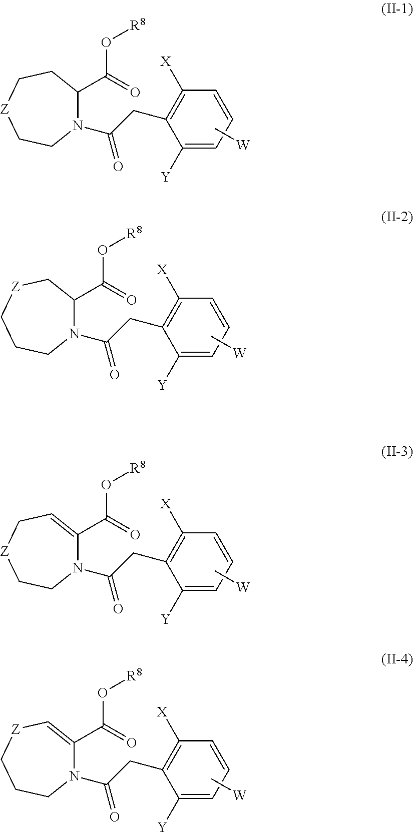

in which W, X, Y and Z have the meanings given above and R⁸ represents C₁-C₄-alkyl, preferably methyl or ethyl, or represents an amino, C₁-C₄-alkylamino or C₁-C₄-dialkylamino group, optionally in the presence of a suitable solvent or diluent, using a suitable base, to give the compound of the general formula (I) according to the invention, or

b) reacting a compound of the general formula (Ia)

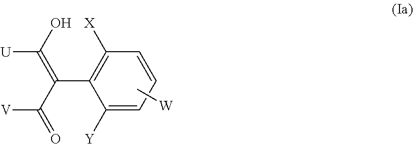

in which U, V, W, X and Y each have the meanings given above, with a compound of the general formula (III)

Hal-L (III)

in which L has the meaning given above and Hal may represent a halogen atom, preferably chlorine or bromine, or may represent a sulfonic acid group, optionally in the presence of a suitable solvent or diluent, and also a suitable base.

The required precursors of the general formulae (II-1), (II-2), (II-3) and (II-4) can also be prepared using synthesis processes known from the literature. Scheme 1 illustrates one of the possible procedures for preparing the precursors (II-1) and (II-3), and the synthesis of precursors (II-2) and (II-4) may, of course, take place in a completely analogous manner

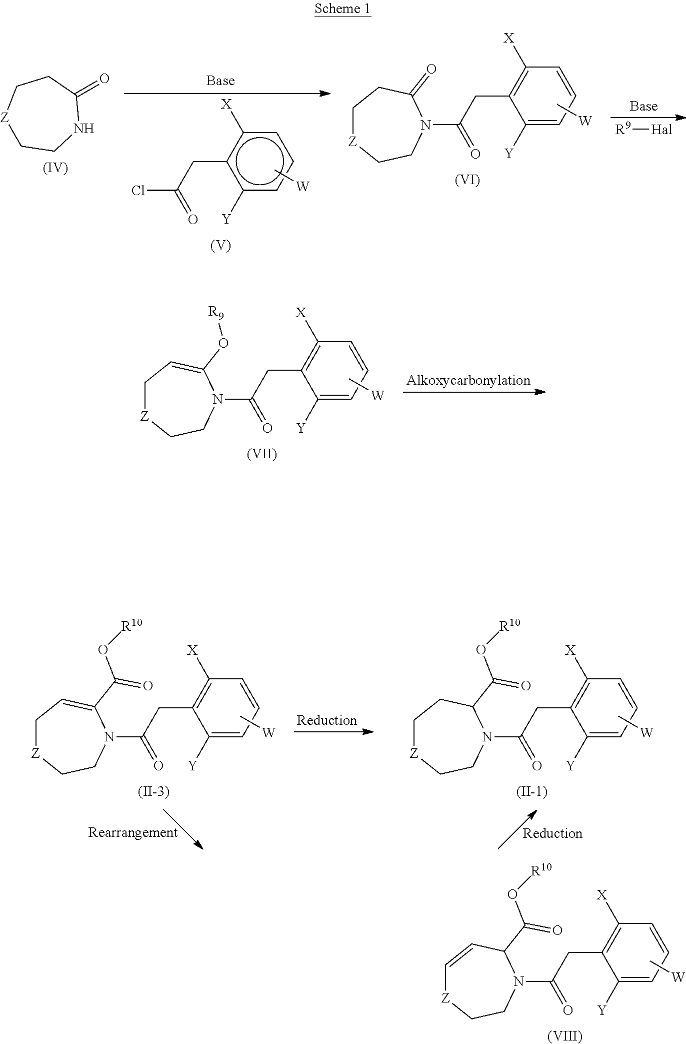

Here, for example, a heterocyclic caprolactam of the general formula (IV) is acylated with a phenylacetyl chloride of the general formula (V), optionally in the presence of a suitable base, to give a compound of the general formula (VI), where Z, X, Y and W each have the meaning given above. Suitable bases are, for example, organometallic reagents such as n-butyllithium, s-butyllithium or lithium diisopropylamide. Compounds of the type (IV) are known or can be prepared analogously to known processes. Phenylacetic acids and their chlorides of the general formula (V) in which X, Y and W have the meaning given above are likewise known from the literature.

For relevant methods see, inter alia, Kobunshi Kagaku 1970, 27 (297), 1-20 or U.S. Pat. No. 2,771,468 and the laid-open patents cited at the outset.

For the conversion of the intermediate (VI) into compounds of type (VII) in which X, Y, W and Z correspond to the definition described above and R⁹ may represent, for example, a phosphonic ester group such as —PO(OMe)₂, —PO(OEt)₂ or —PO(OC₆H₅)₂ or a sulfonic ester group such as methylsulfonyl, phenylsulfonyl or trifluoromethylsulfonyl, the presence of a suitable base such as, for example, potassium hexamethyldisilazane, n-butyllithium or lithium diisoproylamide may be advantageous. Otherwise, such reactions can be carried out in close analogy to the prior art. Details are described, for example, in J. Org. Chem., 60(9), 2656-7; 1995 or Bioorg & Med. Chemistry Letters, 17(21), 5872-5875; 2007); Eur. J. Org. Chem., (7), 1306-1317; 2013; Synlett, (6), 913-916; 2009 or Chem. Commun. 16, 1757-1758; 1998.

The precursor of the general formula (II-3) required for process a) is obtained by alkoxycarbonylation of the intermediates of the general formula (VII). Such reaction methods are generally known and can be carried out analogously to methods known from the literature, see, for example, Eur. J. Org. Chem., (7), 1306-1317; 2013 or Synlett, (6), 913-916; 2009.

For reducing precursors of the general formula (II-3) to the optionally desired precursors of the general formula (II-1), there is likewise available a large number of methods and reducing agents known from the literature. Also obvious is, for example, the catalytic hydrogenation using customary transition metal catalysts such as, for example, palladium or nickel in suitable standard solvents such as methanol, ethanol or ethyl acetate.

For the execution of the catalytic hydrogenation, it may in some cases be advantageous to initially convert the intermediate of the general formula (II-3) with a suitable base into its isomer (VIII) and then subject this to the reduction described above.

The rearrangement of the double bond is effected by treatment with bases, for example potassium tert-butoxide, potassium hexamethyltdisilazide or lithium diisopropylamide, in an inert solvent, preferably at low temperatures, according to processes known from the literature. For further details of this technique see, for example, J. Am. Chem. Soc., 108(23), 7373-7377; 1986; US 6413448; Tetrahedron 55(12), 3791-3802; 199; Tetrahedron, 66(45), 8605-8614; 2010.

Alternatively, the preparation of the precursors (II-1) to (II-4) can also take place by the route illustrated in Scheme 2 for compounds (II-2) and (II-4). Here, starting materials of the general formula (IX) in which Z has the meaning given above are initially converted into compounds of the general formula (X), where PG represents a suitable NH protective group such as, for example, benzyl, benzyloxycarbonyl, phenylcarbamoyl or formyl. Analogously to the above-described methods, these intermediates can then be converted into the compounds of the general formulae (II-2) and (II-4).

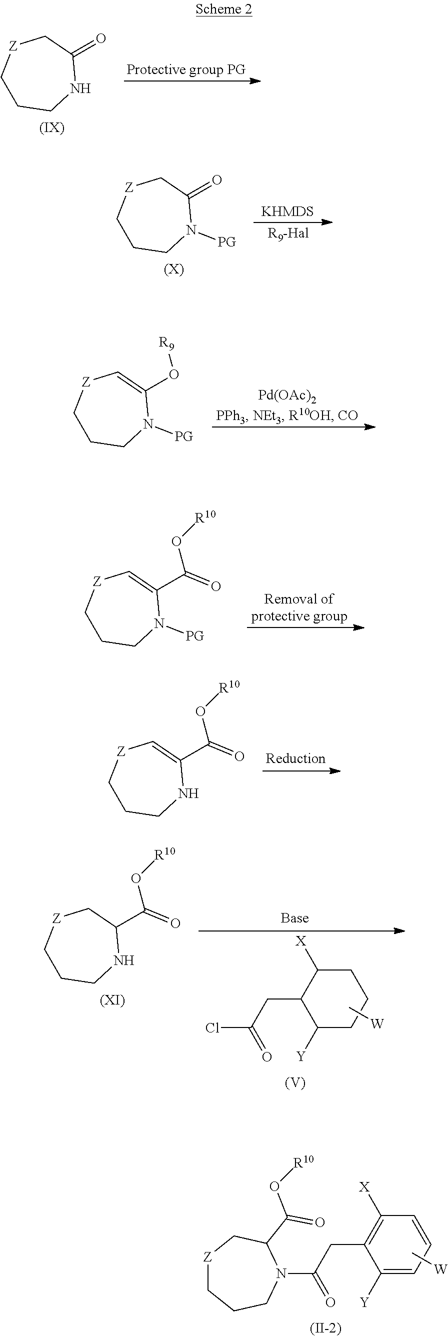

The compounds according to the invention of the formula (I) (and/or salts thereof), referred to hereinbelow together as “compounds according to the invention”, have an excellent herbicidal effectiveness against a broad spectrum of economically important mono- and dikotyledonous annual weeds. The active compounds also act efficiently on perennial weeds which produce shoots from rhizomes, root stocks and other perennial organs and which are difficult to control.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

If the compounds of the invention are applied to the soil surface before germination, either the emergence of the weed seedlings is prevented completely or the weeds grow until they have reached the cotyledon stage, but then they stop growing and ultimately die completely after three to four weeks have passed.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner

Although the compounds according to the invention have an excellent herbicidal activity towards mono-and dikotyledonous weeds, crop plants of economically important crops e.g. dicotyledonous crops of the genera Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, are damaged only insignificantly, or not at all, depending on the structure of the particular compound according to the invention and its application rate. For these reasons, the present compounds are very suitable for selective control of unwanted plant growth in plant crops such as agriculturally useful plants or ornamental plants.

In addition, the compounds of the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. In addition, they are also suitable for general control and inhibition of unwanted vegetative growth without killing the plants. An inhibition of the vegetative growth plays a large role in many mono- and dikotyledonous crops since, for example, the storage formation can be reduced or completely prevented as a result.

By virtue of their herbicidal and plant growth regulatory properties, the active compounds can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other particular properties relate, for example, to the harvested material with regard to quantity, quality, storability, composition and specific constituents. For instance, there are known transgenic plants with an elevated starch content or altered starch quality, or those with a different fatty acid composition in the harvested material.

As regards transgenic crops, preference is given to the application of the compounds according to the invention in economically important transgenic crops of useful plants and ornamental plants, e.g. of cereals such as wheat, barley, rye, oats, millet, rice, maniok and corn or else crops of sugar cane, cotton, soybean, rapeseed, potatos, tomatoes, peas and other vegetable varieties. Preferably, the compounds of the invention can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with modified properties can be generated with the aid of recombinant methods (see, for example, EP-A-0221044, EP-A-0131624). For example, there have been descriptions in several cases of:
genetic modifications of crop plants for the purpose of modifying the starch synthesized in the plants (for example WO 92/11376, WO 92/14827, WO 91/19806),
transgenic crop plants which are resistant to particular herbicides of the glufosinate type (cf., for example, EP-A-0242236, EP-A-242246) or glyphosate type (WO 92/00377) or the sulfonylurea type (EP-A-0257993, U.S. Pat. No. 5,013,659),
transgenic crop plants, for example cotton, with the ability to produce Bacillus thuringiensis toxins (Bt toxins), which make the plants resistant to particular pests (EP-A-0142924, EP-A-0193259),
transgenic crop plants having a modified fatty acid composition (WO 91/13972),
genetically modified crop plants with novel constituents or secondary metabolites, for example novel phytoalexins, which bring about an increased disease resistance (EPA 309862, EPA0464461),
genetically modified plants having reduced photorespiration, which have higher yields and higher stress tolerance (EPA 0305398),
transgenic crop plants which produce pharmaceutically or diagnostically important proteins (“molecular pharming”),
transgenic crop plants which feature higher yields or better quality,
transgenic crop plants which feature a combination, for example, of the abovementioned novel properties (“gene stacking”).

Numerous molecular biology techniques which can be used to produce novel transgenic plants with modified properties are known in principle; see, for example, I. Potrykus and G. Spangenberg (eds.) Gene Transfer to Plants, Springer Lab Manual (1995), Springer Verlag Berlin, Heidelberg, or Christou, “Trends in Plant Science” 1 (1996) 423-431.

For such recombinant manipulations, nucleic acid molecules which allow mutagenesis or sequence alteration by recombination of DNA sequences can be introduced into plasmids. With the aid of standard methods, it is possible, for example, to undertake base exchanges, remove parts of sequences or add natural or synthetic sequences. To join the DNA fragments with one another, adapters or linkers can be placed onto the fragments, see, for example, Sambrook et al., 1989, Molecular Cloning, A Laboratory Manual, 2nd edition, Cold Spring Harbor Laboratory Press, Cold Spring Harbor, N.Y., or Winnacker “Gene and Klone” [Genes and clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227, Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

The compounds of the invention can be used with preference in transgenic crops which are resistant to growth regulators, for example dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active compounds.

When the active compounds of the invention are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also provides for the use of the compounds of the invention as herbicides for control of harmful plants in transgenic crop plants.

In a preferred embodiment of the present invention, the compounds of the general formula (I) can also be used to control those harmful plants e.g. from the group Agrostis, Alopecurus, Apera, Avena, Brachiaria, Bromus, Cenchrus, Digitaria, Echinochloa, Eleusine, Eriochloa, Leptochloa, Lolium, Ottochloa, Panicum, Pennisetum, Phalaris, Poa, Rottboellia, Setaria and/or Sorghum weeds; in particular Alopecurus, Apera, Avena, Brachiaria, Bromus, Digitaria, Echinochloa, Eriochloa, Lolium, Panicum, Phalaris, Poa, Setaria and/or Sorghum weeds,
which are resistant to one or more herbicides inhibiting the enzyme acetyl-CoA-carboxylase (ACCase). ACCase-inhibiting herbicides are, inter alia, pinoxaden, clodinafop-propargyl, fenoxaprop-P-ethyl, diclofop-methyl, fluazifop-P-butyl, haloxyfop-P-methyl, quizalofop-P-ethyl, propaquizafop, cyhalofop-butyl, clethodim, sethoxydim, cycloxydim, tralkoxydim or butroxydim;
and/or are resistant to glyphosate,
and/or are resistant to one or more herbicides inhibiting the acetolactate synthase (ALS), such as, for example, one or more sulfonylurea herbicides (e.g. iodosulfurone-methyl, mesosulfurone-methyl, tribenuron-methyl, triasulfurone, prosulfurone, sulfosulfurone, pyrazosulfurone-ethyl, bensulfurone-methyl, nicosulfurone, flazasulfurone, iofensulfurone, metsulfurone-methyl, or any other sulfonylurea disclosed in the “The Pesticide Manual”, 15th edition (2009) or 16th edition (2012), C. D. S. Tomlin, British Crop Protection Council, and/or one or more triazolopyrimidine herbicides (e.g. florasulam, pyroxsulam or penoxsulam) and/or one or more pyrimidinyl (thio or oxy) benzoate herbicides (e.g. bispyribac-sodium or pyriftalid) and/or one or more sulfonylamino-carbonyltriazolinone herbicides (e.g. thiencarbazone-methyl, propoxycarbazone-sodium or flucarbazone-sodium) and/or imidazolinone herbicides (e.g. imazamox).

Specific examples of such harmful grasses resistant to ACCase and/or ALS inhibitors and/or glyphosate are, inter alia, Alopecurus myosuroides, Apera spica-venti, Avena fatua, Avena sterilis, Brachiaria decumbens, Brachiaria plantaginea, Digitatia horizontalis, Digitaria insularis, Digitaria sanguinalis, Echinochloa colona, Echinochloa crus-galli, Eleusine indica, Lolium multiflorum, Lolium rigidum, Lolium perenne, Phalaris minor, Phalaris paradoxa, Setaria viridis, Setaria faberi or Setaria glauca.

In a particularly preferred embodiment of the present invention, the compounds according to the invention of the general formula (I) can be used against harmful plants
which are resistant to one or more ACCase inhibiting herbicides (e.g. selected from the above list) and indeed at least partially on account of mutations (e.g. substitution) of one or more amino acids in the ACCase target site of the harmful plant (cf. e.g. S. B. Powles and Qin Yu, “Evolution in Action: Plants Resistant to Herbicides”, Annu. Rev. Plant Biol., 2010, 61, p. 317-347); and/or
which are resistant to glyphosate, and indeed at least partly on account of mutation (e.g. substitution) of one or more amino acids at the EPSPS target site in the weed in question to which glyphosate is directed; and/or
which are resistant to one or more ALS-inhibiting herbicides (e.g. selected from the above list of ALS-inhibiting herbicides) and indeed at least partly on account of mutations (e.g. substitution) of one or more amino acids in the ALS target site in the weed in question (cf. e.g. S. B. Powles and Qin Yu, “Evolution in Action: Plants Resistant to Herbicides”, Annu. Rev. Plant Biol., 2010, 61, p. 317-347); and/or
which are resistant to one or more ACCase inhibiting herbicides (e.g. selected from the above list) and/or to glyphosate and/or to one or more ALS-inhibiting herbicides (e.g. selected from the above list) and indeed at least partially through a metabolically induced herbicide resistance, e.g. at least partially due to a cytochrome P450-mediated metabolism (cf. e.g. S. B. Powles and Qin Yu, “Evolution in Action: Plants Resistant to Herbicides”, Annu. Rev. Plant Biol., 2010, 61, p. 317-347).

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds according to the invention can be formulated in various ways according to which biological and/or chemical physical parameters are pregiven. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of micro granules, spray granules, absorption and adsorption granules, water-dispersible granules (WG), water-soluble granules (SG), ULV formulations, microcapsules and waxes.

These individual formulation types are known in principle and are described, for example, in: Winnacker Kuchler, “Chemische Technologic [Chemical Technology]”, Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”, 2nd Ed., J. Wiley & Sons, N.Y.; C. Marsden, “Solvents Guide”, 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964, Schonfeldt, “Grenzfláchenaktive Áthylenoxidaddukte [Interface-active ethylene oxide adducts]”, Wiss. Verlagsgesell., Stuttgart 1976, Winnacker Kuchler, “Chemische Technologic [Chemical Technology]”, Volume 7, C. Hanser Verlag Munich, 4th Ed. 1986.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix Suitable safeners are, for example, mefenpyr-diethyl, cyprosulfamide, isoxadifen-ethyl, cloquintocet-mexyl and dichlormid.

Wettable powders are preparations uniformly dispersible in water which, alongside the active compound apart from a diluent or inert substance, also comprise surfactants of an ionic and/or non-ionic type (wetting agent, dispersant), e.g. polyoxyethylated alkylphenols, polyoxethylated fatty alcohols, polyoxethylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active compounds are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonic acid salts such as Ca dodecylbenzenesulfonate or non-ionic emulsifiers such as fatty acid polyglycol esters, alkylaryl polyglycol ethers, fatty alcohol polyglycol ethers, propylene oxide ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates can be based on water or oil. They may be prepared, for example, by wet-grinding by means of commercial bead mills and optional addition of surfactants as have, for example, already been listed above for the other formulation types.

Emulsions, e.g. oil-in-water emulsions (EW), can be prepared, for example, by means of stirrers, colloid mills and/or static mixers using aqueous organic solvents and optionally surfactants, as have already been listed e.g. above for the other formulation types.

Granules can be produced either by spraying the active compound onto adsorptive granular inert material or by applying active compound concentrates to the surface of carriers, such as sand, kaolinites or granular inert material, by means of adhesives, for example polyvinyl alcohol, sodium polyacrylate or else mineral oils. Suitable active compounds can also be granulated in the manner customary for producing fertilizer granules—if desired in a mixture with fertilizers.

Water-dispersible granules are usually produced by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray-Drying Handbook” 3rd Ed. 1979, G. Goodwin Ltd., London; J. E. Browning, “Agglomeration”, Chemical and Engineering 1967, pages 147 ff; “Perry's Chemical Engineer's Handbook”, 5th Ed., McGraw Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations generally comprise 0.1 to 99% by weight, in particular 0.1 to 95% by weight, of compounds according to the invention.

In wettable powders, the active compound concentration is e.g. about 10 to 90% by weight, the remainder to 100% by weight consists of customary formulation constituents. In the case of emulsifiable concentrates, the active compound concentration can be about 1 to 90, preferably 5 to 80% by weight. Dust-type formulations contain 1 to 30% by weight of active compound, preferably at most 5 to 20% by weight of active compound, sprayable solutions comprise about 0.05 to 80, preferably 2 to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) varies with the external conditions, including, inter alia, temperature, humidity and the type of herbicide used. It can vary within wide limits, for example between 0.001 and 1.0 kg/ha or more of active substance, but it is preferably between 0.005 and 750 g/ha.

The examples below illustrate the preparation of the compounds according to the invention:

### A. CHEMICAL EXAMPLES

### Example Ia-1-3

### 8-(2,6-Diethyl-4-methylphenyl)-9-hydroxy-1,4,5,9a-tetrahydropyrrolo[1,2-d][1,4]oxazepin-7(2H)-one

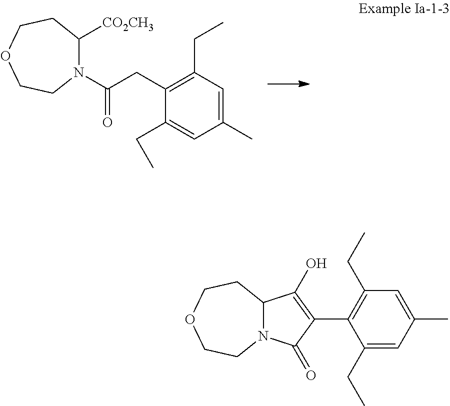 [SMILES: CCc1cc(C)cc(CC)c1CC(=O)N1CCOCCC1C(=O)OC ; CCc1cc(C)cc(CC)c1C1=C(O)C2CCOCCN2C1=O]

0.4 g (3.6 mmol) of potassium tert-butoxide are dissolved in 12 ml of DMF, and at 70° C. a solution of 0.50 g (1 4 mmol) of methyl 4-[(2,6-diethyl-4-methylphenyl)acetyl]-1,4-oxazepane-5-carboxylate in 12 ml of DMF is added dropwise over 60 min. The mixture is stirred at 70° C. for another 10 min and the progress of the reaction is monitored by HPLC. Subsequently, the mixture is stirred with 100 ml of ice water and adjusted to pH=1 with 2N hydrochloric acid. The solvent is distilled off, the residue is then stirred with 15 ml of water and 100 ml of dichloromethane and the organic phase is separated off. Drying over sodium sulfate and distillative removal of the solvent gives 0.43 g of the title compound.

The following compounds according to the invention were prepared analogously to Example Ia-1-3:

TABLE 1

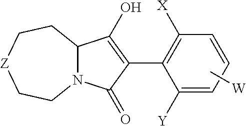

| Example | | | | | |
|---|---|---|---|---|---|
| No | Z | X | W | Y | ¹H-NMR (400 MHz, d₆-DMSO |
| Ia-1-1 | O | Me | 4-Me | Me | δ = 1.80 (mc, 1H), 2.02 (s, 3H), 2.05 (s, 3H), 2.20 (s, 3H), 2.23 (mc, 1H), 3.19 (mc, 1H), 3.55- |
| | | | | | 3.79 (m, 5H), 4.30 (mc, 1H), 6.83 (s, 2H), 10.80 (s, br, 1H) |
| Ia-1-2 | O | Me | 4-Me | Et | δ = 1.08 (mc, 3H), 1.82 (mc, 1H), 2.02 (s, 3H), 2.03 (s, 3H), 2.22 (mc, 1H), 2.38 (mc, 2H), 3.20 |
| | | | | | (mc, 1H), 3.50-3.80 (m, 5H), 4.31 (mc, 1H), 6.85 (mc, 2H), 10.79 (s, br, 1H) |
| Ia-1-3 | O | Et | 4-Me | Et | δ = 1.00 (mc, 6H), 1.81 (mc, 1H), 2.23 (mc, 1H), 2.25 (s, 3H), 2.32 (mc, 2H), 3.20 (mc, 1H), |
| | | | | | 3.51-3.80 (m, 5H), 4.32 (mc, 1H), 6.88 (mc, 2H), 10.75 (s, br, 1H) |
| Ia-1-4 | O | Et | 4-H | Me | δ = 1.01 (mc, 3H), 1.82 (mc, 1H), 2.09 (mc, 3H), 2.25 (mc, 1H), 2.40 (mc, 2H), 3.21 (mc, 1H), |
| | | | | | 3.55-3.80 (m, 5H), 4.32 (mc, 1H), 7.03 (mc, 2H), 7.12 (mc, 1H), 10.85 (s, br, 1H) |
| Ia-1-5 | O | H | 4-Me | MeO | δ = 1.70 (mc, 1H), 2.17-2.23 (m, 1H), 2.30 (s, 3H), 3.20 (mc, 1H), 3.60-3.79 (m, 5H), 3.70 (s, |
| | | | | | 3H), 4.23 (mc, 1H), 6.72 (d, 1H), 6.80 (s, 1H), 7.08 (d, 1H), 10.40 (s, br, 1H) |
| Ia-1-6 | O | Et | 4-Cl | MeO | δ = 1.02 (mc, 3H), 1.70-1.85 (m, 1H), 2.16-2.25 (m, 1H), 2.42 (mc, 2H), 3.20 (mc, 1H), 3.50-3.78 |
| | | | | | (m, 5H), 3.68 (mc, 3H), 4.29 (mc, 1H), 6.89 (mc, 2H) |
| Ia1-7 | O | Et | 4-Cl | EtO | δ = 1.02 (dt, 3H), 1.21 (t, 3H), 2.18-2.25 (m, 1H), 2.44 (q, 2H), 3.20-3.26 (m, 1H), 3.49-3.98 (m, |
| | | | | | 8H), 4.27-4.28 (m, 1H), 6.84-6.88 (m, 2H) |
| Ia-1-8 | O | Cl | 4-Me | MeO | δ = 1.80 (mc, 1H), 2.19 (mc, 1H), 2.30 (s, 3H), 3.18 (mc, 1H), 3.53-3.75 (m, 5H), 3.70 (mc, 3H), |
| | | | | | 4.28 (mc, 1H), 6.80 (mc, 1H), 6.88 (mc, 1H), 10.88 (s, br, 1H) |
| Ia-1-9 | O | Me | 4-Br | Me | δ = 1.79-1.83 (m, 1H), 2.07 (s, 3H), 2.10 (s, 3H), 2.23-2.27 (m, 1H), 3.19-3.24 (m, 1H), 3.58-3.76 |
| | | | | | (m, 5H), 4.35 (mc, 1H), 7.26 (s, 1H) |
| Ia-1-10 | O | Et | 4-Br | Me | δ = 1.02 (dt, 3H), 1.80 (mc, 1H), 2.08 (d, 3H), 2.25 (mc, 1H), 2.43 (q, 2H), 3.22 (mc, 1H), 3.50- |
| | | | | | 3.76 (m, 5H), 7.25 (d, 2H), 7.95 (s, 1H) |
| Ia-1-11 | O | Et | 4-Br | Et | δ = 1.03 (dt, 6H), 1.80 (mc, 1H), 2.25 (mc, 1H), 2.41 (q, 4H), 3.24 (mc, 1H), 3.65-3.77 (m, 5H), |
| | | | | | 4.36 (mc, 1H), 7.26 (s, 2H), 7.95 (s, 1H) |
| Ia-1-12 | O | Me | 4-Me | Br | |
| Ia-1-13 | O | Et | 4-Me | Br | δ = 1.03 (dt, 3H), 1.84-1.99 (m, 1H), 1.21-1.25 (m, 1H), 2.33 (s, 3H), 2.42 (q, 2H), 3.19-3.23 (m, |
| | | | | | 1H), 3.54-3.76 (m, 5H), 4.32 (t, 1H), 7.06 (s, 1H), 7.31 s, 1H) |
| Ia-1-14 | O | Me | 4-Me | Cl | |
| Ia-1-15 | O | Et | 4-Me | Cl | |
| Ia-1-16 | O | Me | 4-C≡Me | Me | δ = 1.78-1.90 (m, 1H), 2.01 (s, 3H), 2.03 (s, 3H), 2.06 (s, 3H), 2.23-2.30 (m, 1H), 3.19-3.22 (m, |
| | | | | | 1H), 3.58-3.76 (m, 5H), 4.34 (mc, 1H), 7.07 (s, 2H) |
| Ia-1-17 | O | Et | 4-C≡Me | Me | δ = 1.03 (dt, 3H), 1.75-1.86 (m, 1H), 2.23-2.30 (m, 1H), 2.41 (q, 2H), 3.20-3.31 (m, 1H), 3.58- |
| | | | | | 3.76 (m, 5H), 4.34 (t, 1H), 7.07 (d, 2H) |
| Ia-1-18 | O | Et | 4-C≡Me | Et | δ = 1.03 (dt, 6H), 1.82 (mc, 1H), 2.05 (s, 3H), 2.25 (mc, 1H), 2.39 (q, 4H), 3.21 (mc, 1H), 3.56- |
| | | | | | 3.78 (m, 5H), 4.36 (mc, 1H), 7.10 (s, 2H) |
| Ia-1-19 | O | Me | 4-C≡Me | OMe | δ = 1.75-1.1.83 (m, 1H), 1.91 (s, 3H), 2.04 (s, 3H), 2.20-2.32 (m, 1H), 3.16-3.21 (m, 1H), 3.59- |
| | | | | | 3.73 (m, 5H), 4.27-4.30 (m, 1H), 6.79-6.86 (m, 2H) |
| Ia-1-20 | O | Et | 4-C≡Me | OMe | |
| Ia-1-21 | O | MeO | 4-C≡Me | F | |
| Ia-1-22 | O | MeO | 4-C≡Me | MeO | |
| Ia-1-23 | O | MeO | 4-C≡Me | H | |
| Ia-1-24 | O | MeO | 4-C≡Me | Cl | δ = 1.78-1.86 (m, 1H), 1.91 (s, 3H), 1.99 (s, 3H), 2.07 (s, 3H), 2.18-2.25 (m, 1H), 3.15-3.22 (m, |
| | | | | | 1H), 3.61-3.74 (m, 5H), 4.30 (t, 1H), 6.96-7.06 (m, 2H) |
| Ia-1-25 | O | Me | 4-OCH₂CF₃ | Me | δ = 1.75-1.88 (m, 1H), 2.07 (s, 3H), 2.09 (s, 3H), 2.20-2.39 (m, 1H), 3.20-3.32 (m, 1H), 3.59-3.76 |
| | | | | | (m, 5H), 4.32 (dt, 1H), 4.71 (q, 2H), 6.76 (s, 2H) |
| Ia-1-26 | O | Et | 4-OCH₂CF₃ | Me | δ = 1.09 (dt, 3H), 1.78-1.85 (m, 1H), 2.05 (d, 3H), 2.20-2.28 (m, 1H), 2.35 (q, 2H), 3.17-3.22 (m, |
| | | | | | 1H), 3.54-3.78 (m, 5H), 4.34 (mc, 1H), 4.73 (q, 2H), 6.76 (d, 2H) |
| Ia-1-27 | O | Et | 4-OCH₂CF₃ | Et | δ = 1.01 (dt, 6H), 1.80-1.85 (m, 1H), 2.22-2.26 (m, 1H), 2.39 (q, 4H), 3.19-3.24 (m, 1H), 3.53- |
| | | | | | 3.77 (m, 5H), 4.33 (mc, 1H), 4.75 (q, 2H), 6.76 (s, 2H) |
| Ia-1-28 | O | Br | 4-Cl | Et | |
| Ia-1-29 | O | MeO | 4-Me | Me | |
| Ia-1-30 | O | Me | 4-(4-ClC₆H₄) | Me | |
| Ia-1-31 | O | Me | 4-(4-ClC₆H₄) | F | |
| Ia-1-32 | O | Me | 4-(4-ClC₆H₄) | Cl | |
| Ia-1-33 | O | Me | 4-(4-FC₆H₄) | Me | |
| Ia-1-34 | O | Me | 4-(4-FC₆H₄) | F | |
| Ia-1-35 | O | Me | 4-(4-FC₆H₄) | Cl | |
| Ia-1-36 | O | MeO | 4-(4-ClC₆H₄) | Me | |
| Ia-1-37 | O | MeO | 4-(4-ClC₆H₄) | F | |
| Ia-1-38 | O | MeO | 4-(4-ClC₆H₄) | Cl | |
| Ia-1-39 | O | MeO | 4-(4-FC₆H₄) | Me | |
| Ia-1-40 | O | MeO | 4-(4-FC₆H₄) | F | |
| Ia-1-41 | O | MeO | 4-(4-FC₆H₄) | Cl | |
| Ia-1-42 | O | Me | 5-(4-ClC₆H₄) | H | |
| Ia-1-43 | O | Me | 5-(4-FC₆H₄) | H | |
| Ia-1-44 | O | MeO | 5-(4-ClC₆H₄) | H | |
| Ia-1-45 | O | MeO | 5-(4-FC₆H₄) | H | |
| Ia-1-46 | O | F | 5-(4-ClC₆H₄) | H | |
| Ia-1-47 | O | F | 5-(4-FC₆H₄) | H | |
| Ia-1-48 | O | Cl | 5-(4-ClC₆H₄) | H | |
| Ia-1-49 | O | Cl | 5-(4-FC₆H₄) | H | |
| Ia-1-50 | O | Br | 5-(4-BrC₆H₄) | H | |
| Ia-1-51 | O | Br | 5-(4-ClC₆H₄) | H | |
| Ia-1-52 | O | Br | 5-(4-FC₆H₄) | H | |

TABLE 2

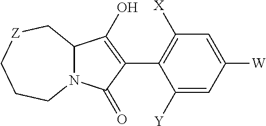

| Example | | | | | |
|---|---|---|---|---|---|
| No. | Z | X | W | Y | ¹H-NMR (400 MHz, d₆-DMSO) |
| Ia-2-1 | O | Me | Me | Me | δ = 1.63 (mc, 2H), 2.05 (s, 6H), 2.23 (mc, 5H), 3.39 (t, 2H), 3.57 (t, 2H), 4.45 (br s, 1H), 6.88 (s, 2H), 10.92 (br |
| | | | | | s, 1H) |
| Ia-2-2 | O | Et | Me | Et | δ = 1.00 (t, 6H), 1.64 (quint, 2H), 2.28 (s, 3H), 2.34 (mc, 4H), 3.38 (mc, 2H), 3.58 (mc, 2H), 4.44 (mc, 1H), |
| | | | | | 4.90 (s, 1H), 5.08 (s, 1H), 6.90 (s, 2H) |

TABLE 3

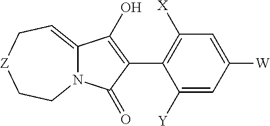

| Example | | | | | |
|---|---|---|---|---|---|
| No | Z | X | W | Y | ¹H-NMR (400 MHz, d₆-DMSO) |
| Ia-3-1 | O | Me | Me | Me | δ = 2.05 (s, 6H), 2.24 (s, 3H), 3.67 (mc, 2H), 3.86 (mc, 2H), 4.44 (d, 2H), 6.64 (mc, 1H), 6.88 (s, 2H), 10.91 (br |
| | | | | | s, 1H) |
| Ia-3-2 | O | Me | Me | Et | δ = 1.01 (t, 3H), 2.03 (s, 3H), 2.26 (s, 3H), 2.37 (q, 2H), 3.68 (mc, 2H), 3.86 (mc, 2H), 4.54 (d, 2H), 5.63 (mc, |
| | | | | | 1H), 6.89 (s, 2H), 10.89 (s, 1H) |
| Ia-3-3 | O | Et | Me | Et | δ = 1.01 (t, 3H), 2.28 (s, 3H), 2.35 (q, 4H), 3.67 (mc, 2H), 3.86 (mc, 2H), 4.45 (d, 2H), 5.63 (mc, 1H), 6.91 (s, |
| | | | | | 2H), 10.88 (s, 1H) |
| Ia-3-4 | O | Et | Cl | MeO | δ = 1.03 (t, 3H), 2.39 (q, 2H), 3.69 (s, 3H), 3.85 (mc, 2H), 4.44 (d, 2H), 5.61 (mc, 1H), 6.92 (s, 2H), 10.90 |
| | | | | | (s, 1H) |
| Ia-3-5 | O | Cl | Me | MeO | δ = 2.29 (s, 3H), 3.65 (mc, 2H), 3.70 (s, 2H), 3.84 (mc, 2H), 4.43 (d, 2H), 5.60 (mc, 1H), 6.83 (s, 1H), 6.91 |
| | | | | | (s, 1H) |
| Ia-3-6 | O | Me | Br | Me | δ = 2.03 (s, 6H), 3.67 (mc, 2H), 3.86 (d, 2H), 6.68 (mc, 1H), 7.30 (s, 2H), 11.12 (s, 1H) |

### Example Ib-1-1

### 8-(2,6-Diethyl-4-methylphenyl)-7-oxo-1,2,4,5,7,9a-hexahydropyrrolo[1,2-d][1,4]oxazepin-9-yl methyl carbonate

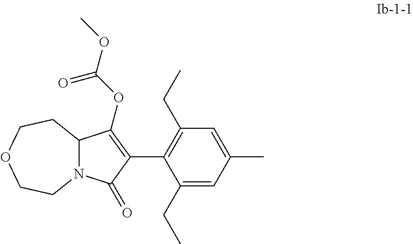 [SMILES: CCc1cc(C)cc(CC)c1C1=C(OC(=O)OC)C2CCOCCN2C1=O]

0.32 g (1.01 mmol) of 8-(2,6-diethyl-4-methylphenyl)-9-hydroxy-1,4,5,9a-tetrahydropyrrolo[1,2-d][1,4]oxazepin-7(2H)-one (Example Ia-1-3) are initially charged in 30 ml of dichloromethane, and 0.41 g (4.1 mmol) of triethylamine is added. 0.11 g (1 1 mmol) of methyl chloroformate is added at room temperature and the mixture is stirred at this temperature for another 20 h. Subsequently, 10 ml of water are added and the organic phase is separated off. Drying (sodium sulfate), distillative removal of the solvent and chromatography on silica gel give 0.31 g of the target compound as a light-yellow oil.

The following compounds according to the invention were prepared analogously to Example Ib-1-1:

TABLE 4

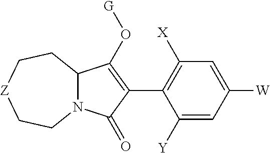

| Example | | | | | | |
|---|---|---|---|---|---|---|
| No. | G | Z | X | W | Y | ¹H-NMR (400 MHz, CDCl₃) |
| Ib-1-1 | —CO₂CH₃ | O | Et | Me | Et | δ = 1.12 (mc, 6H), 1.90 (mc, 1H), 2.23 (mc, 1H), 2.32 (s, 3H), 2.36-2.52 (mc, |
| | | | | | | 4H), 3.40 (mc, 1H), 3.79 (s, 3H), 3.75-3.92 (m, 4H), 4.15 (mc, 1H), 4.88 (mc, |
| | | | | | | 1H), 6.91 (mc, 2H) |
| Ib-1-2 | —CO₂CH₂CH₃ | O | Et | Me | Et | δ = 1.12 (mc, 6H), 1.25 (t, 3H), 1.91 (mc, 1H), 2.23 (mc, 1H), 2.31 (s, 3H), 2.35- |
| | | | | | | 2.55 (mc, 4H), 3.41 (mc, 1H), 3.80 (s, 3H), 3.78-3.91 (m, 4H), 4.10-4.22 (m, 3H), |
| | | | | | | 4.89 (mc, 1H), 6.91 (mc, 2H) |
| Ib-1-3 | tBuCO— | O | Et | Me | Et | δ = 1.06 (s, 9H), 1.10 (mc, 6H), 1.87 (mc, 1H), 2.25 (mc, 1H), 2.30 (s, 3H), 2.35- |
| | | | | | | 2.53 (mc, 4H), 3.78-3.93 (m, 4H), 4.83 (mc, 1H), 6.88 (mc, 2H) |
| Ib-1-4 | CH₃CO— | O | Et | Me | Et | δ = 1.10 (mc, 6H), 2.30 (s, 3H), 3.78 (s, 3H), 4.88 (mc, 1H), 6.92 (mc, 2H) |
| Ib-1-5 | —CO₂CH₂CH₂OCH₃ | O | Et | Me | Et | δ = 1.11 (t, 3H), 1.13 (t, 3H), 1.80-1.90 (m, 1H), 2.18-2.27 (m, 1H), 2.32 (s, 3H), |
| | | | | | | 2.44 (mc, 4H), 2.61 (q, 2H), 3.23 (s, 3H), 3.42 (dd, 1H), 3.55 (q, 2H), 3.77-3.88 |
| | | | | | | (m, 4H), 4.10-4.19 (m, 1H), 4.90 (dd, 1H), 6.92 (s, 2H) |
| Ib-1-6 | —CO₂CH₃ | O | Et | Me | Me | δ = 1.11 (t, 3H), 1.91 (mc, 1H), 2.13 (s, 3H), 2.25 (mc, 1H), 2.29 (s, 3H), 2.38- |
| | | | | | | 2.55 (m,2H), 3.77 (s, 3H), 4.81 (mc, 1H), 6.90 (mc, 2H) |
| Ib-1-7 | —CO₂CH₂CH₃ | O | Et | Me | Me | δ = 1.11 (mc, 3H), 1.23 (mc, 3H), 2.13 (s, 3H), 2.29 (s, 3H), 2.25 (mc, 1H), 2.31 |
| | | | | | | (s, 3H), 2.38-2.53 (m, 2H), 3.78-3.92 (m, 3H), 4.10-4.20 (m, 2H), 4.79 (mc, 1H), |
| | | | | | | 6.92 (mc, 2H) |
| Ib-1-8 | tBuCO— | O | Et | Me | Me | δ = 1.07 (s, 9H), 1.10 (mc, 3H), 2.21 (s, 3H), 2.24 (s, 3H), 2.28 (s, 3H), 2.33-2.52 |
| | | | | | | (mc, 2H), 3.40 (mc, 1H), 3.76-3.90 (m, 4H), 4.10-4.20 (m, 2H), 4.81 (mc, 1H), |
| | | | | | | 6.90 (mc, 2H) |
| Ib-1-9 | CH₃CO— | O | Et | Me | Me | δ = 1.10 (mc, 3H), 2.10 (s, 3H), 2.12 (d, 3H), 2.29 (s, 3H), 2.38-2.55 (mc, 4H), |
| | | | | | | 3.75-3.92 (m, 4H), 4.81 (mc, 1H), 6.91 (mc, 2H) |
| Ib-1-10 | —CO₂CH₂CH₃ | O | Me | Me | Me | δ = 1.23 (mc, 3H), 1.93 (mc, 1H), 2.16 (s, 3H), 2.17 (s, 3H), 2.26 (s, 3H), 3.40 |
| | | | | | | (mc, 1H), 4.13 (mc, 2H), 4.79 (mc, 1H), 6.88 (s, 2H) |
| Ib-1-11 | tBuCO— | O | Me | Me | Me | δ = 1.09 (s, 9H), 2.12 (s, 3H), 2.13 (s, 3H), 2.25 (s, 3H), 3.40 (mc, 3H), 4.13 |
| | | | | | | (mc, 1H), 4.80 (mc, 1H), 6.84 (mc, 2H) |
| Ib-1-12 | CH₃CO— | O | Me | Me | Me | δ = 1.88 (mc, 3H), 2.10 (s, 3H), 2.13 (s, 3H), 2.14 (s, 3H), 2.28 (s, 3H), 3.40 |
| | | | | | | (mc, 1H), 3.75- 3.90 (m, 4H), 4.76 (mc, 1H), 6.88 (s, 2H) |
| Ib-1-13 | —CO₂CH₂CH₃ | O | Me | H | Et | δ = 1.12 (t, 3H), 1.22 (t, 3H), 1.95 (mc, 1H), 2.10 (s, 3H), 2.28 (mc, 1H), 2.50 |
| | | | | | | (mc, 2H), 4.18 (mc, 3H), 4.81 (mc, 1H), 7.10 (mc, 2H), 7.21 (mc, 1H) |
| Ib-1-14 | tBuCO— | O | Me | H | Et | δ = 1.07 (s, 9H), 1.12 (t, 3H), 2.18 (d, 3H), 4.15 (mc, 1H), 4.75 (mc, 1H), 7.00- |
| | | | | | | 7.19 (m, 3H) |
| Ib-1-15 | CH₃CO— | O | Me | H | Et | δ = 1.11 (mc, 3H), 2.10 (s, 3H), 2.18 (d, 3H), 4.81 (mc, 1H), 7.10 (mc, 2H), 7.22 |
| | | | | | | (mc, 1H) |
| Ib-1-16 | —CO₂CH₃ | O | Et | Cl | OMe | δ = 1.11 (mc, 3H), 2.50 (mc, 2H), 3.40 (mc, 1H), 3.72 (s, 3H), 3.80 (s, 3H), 4.12 |
| | | | | | | (mc, 1H), 4.80-4.88 (m, 1H), 6.72 (s, 1H), 6.89 (s, 1H) |
| Ib-1-17 | —CO₂CH₂CH₃ | O | Et | Cl | OMe | δ = 1.12 (mc, 3H), 1.27 (mc, 3H), 2.50 (mc, 2H), 4.17 (mc, 2H), 4.80-4.86 (m, |
| | | | | | | 1H), 6.72 (s, 1H), 6.90 (s, 1H) |
| Ib-1-18 | tBuCO— | O | Et | Cl | OMe | δ = 1.12 (s, 9H), 3.73 (d, 3H), 4.74-4.85 (m, 1H), 6.70 (mc, 1H), 6.88 (mc, 1H) |
| Ib-1-19 | —CO₂CH₂CH₃ | O | Et | Cl | EtO | δ = 1.12 (t, 3H), 1.30 (mc, 6H), 1.82-2.05 (m, 1H), 2.20-2.30 (m, 1H), 2.50 (mc, |
| | | | | | | 2H), 3.31-3.43 (m, 1H), 3.72-4.22 (m, 9H), 4.76-5.30 (m, 1H), 6.71 (d, 1H), 6.88 |
| | | | | | | (s, 1H) |
| Ib-1-20 | tBuCO— | O | Et | Cl | EtO | δ = 1.10 (mc, 9H), 1.30 (dt, 3H), 1.75-1.98 (m, 1H), 2.12-2.25 (m, 1H), 2.45-2.64 |
| | | | | | | (m, 4H), 3.32-3.43 (m, 1H), 3.71-3.97 (m, 5H), 4.11-4.16 (m, 1H), 4.75-4.92 (m, |
| | | | | | | 1H), 6.70 (s, 1H), 6.88 (d, 1H) |
| Ib-1-21 | —CO₂CH₃ | O | Cl | Me | OMe | δ = 1.99 (mc, 1H), 2.22 (mc, 1H), 2.31 (s, 3H), 3.77 (d, 3H), 3.81 (s, 3H), 4.82- |
| | | | | | | 4.88 (m, 1H), 6.62 (s, 1H), 6.88 (mc, 1H) |
| Ib-1-22 | —CO₂CH₂CH₃ | O | Cl | Me | OMe | δ = 1.26 (mc, 3H), 2.31 (s, 3H), 3.82 (d, 3H), 4.10-4.25 (m, 3H), 6.61 (s, 1H), |
| | | | | | | 6.89 (mc, 1H) |
| Ib-1-23 | tBuCO— | O | Cl | Me | OMe | δ = 1.18 (s, 9H), 2.32 (s, 3H), 3.72 (s, 3H), 4.80 (mc, 1H), 6.90 (s, 1H), 6.88 |
| | | | | | | (s, 1H) |
| Ib-1-24 | CH₃CO— | O | Cl | Me | OMe | δ = 2.25 (d, 3H), 2.32 (s, 3H), 3.38 (mc, 1H), 3.80 (d, 3H), 4.11 (mc, 1H), 4.80- |
| | | | | | | 4.90 (m, 1H), 6.65 (mc, 1H), 6.85 (mc, 1H) |
| Ib-1-25 | —CO₂CH₃ | O | H | Me | OMe | δ = 1.90 (mc, 1H), 2.21 (mc, 1H), 2.35 (s, 3H), 3.75 (s, 3H), 3.81 (s, 3H), 4.70 |
| | | | | | | (mc, 1H), 6.71 (s, 1H), 6.83 (d, 1H), 7.40 (d, 1H) |
| Ib-1-26 | —CO₂CH₂CH₃ | O | H | Me | OMe | δ = 1.28 (t, 3H), 2.35 (s, 3H), 3.74 (s, 3H), 4.20 (q, 2H), 4.68 (mc, 1H), 6.70 (s, |
| | | | | | | 1H), 6.82 (d, 1H), 7.40 (d, 1H) |
| Ib-1-27 | tBuCO— | O | H | Me | OMe | δ = 1.20 (s, 9H), 2.36 (s, 3H), 3.71 (s, 3H), 4.76 (mc, 1H), 6.70 (s. 1H), 6.82 (d, |
| | | | | | | 1H), 7.27 (d, 1H), 6.70 (s. 1H), 6.82 (d, 1H); 7.27 (d, 1H) |
| Ib-1-28 | CH₃CO— | O | H | Me | OMe | δ = 2.19 (s, 3H), 2.38 (s, 3H), 3.76 (s, 3H), 4.72 (mc, 1H), 6.72 (s. 1H), 6.82 (d, |
| | | | | | | 1H), 7.37 (d, 1H) |
| Ib-1-29 | —CO₂CH₂CH₃ | O | Me | Br | Me | δ = 1.24 (t, 3H), 1.90-1.95 (m, 1H), 2.17 (s, 6H), 2.25-2.30 (m, 1H), 3.38-3.44 |
| | | | | | | (m, 1H), 3.67-3.93 (m, 4H), 4.16 (mc, 3H), 4.76 (dd, 1H), 7.22 (s, 2H) |
| Ib-1-30 | tBuCO— | O | Me | Br | Me | |
| Ib-1-31 | —CO₂CH₂CH₃ | O | Et | Br | Me | |
| Ib-1-32 | tBuCO— | O | Et | Br | Me | |
| Ib-1-33 | —CO₂CH₂CH₃ | O | Et | Br | Et | |
| Ib-1-34 | tBuCO— | O | Et | Br | Et | |
| Ib-1-35 | —CO₂CH₂CH₃ | O | Me | Me | Cl | |
| Ib-1-36 | tBuCO— | O | Me | Me | Cl | |
| Ib-1-37 | —CO₂CH₂CH₃ | O | Et | Me | Cl | |
| Ib-1-38 | tBuCO— | O | Et | Me | Cl | |
| Ib-1-39 | —CO₂CH₂CH₃ | O | Br | Me | Br | |
| Ib-1-40 | tBuCO— | O | Br | Me | Br | |
| Ib-1-41 | —CO₂CH₂CH₃ | O | Et | Me | Br | δ = 1.12 (dt, 3H), 1.28 (dt, 3H), 1.85-1.92 (m, 1H), 2.00-2.10 (m, 1H), 2.23-2.8 |
| | | | | | | (m, 1H), 2.31 (s, 3H), 2.47-2.57 (m, 2H), 3.38-3.46 (m, 1H), 3.79-3.90 (m, 4H), |
| | | | | | | 4.11-4.25 (m, 3H), 4.90 (dt, 1H), 7.02 (s, 1H), 7.29 (s, 1H) |
| Ib-1-42 | tBuCO— | O | Et | Me | Br | δ = 1.12 (mc, 9H), 1.80-2.05 (m, 1H), 2.14-2.19 (m, 1H), 2.31 (s, 3H), 2.55 (mc, |
| | | | | | | 3H), 3.35-3.48 (m, 1H), 3.78-3.90 (m, 4H), 4.10-4.16 (m, 1H), 4.80-4.95 (m, 1H), |
| | | | | | | 7.00 (s, 1H) |
| Ib-1-43 | —CO₂CH₂CH₃ | O | Me | C≡Me | Me | δ = 1.26 (t, 3H), 1.91-1.96 (m, 1H), 2.04 (s, 3H), 2.15 (s, 3H), 2.19 (s, 3H), 2.24- |
| | | | | | | 2.29 (m, 1H), 3.37-3.43 (m, 1H), 3.79-3.91 (m, 5H), 4.14 (q, 2H), 4.76 (mc, 1H), |
| | | | | | | 7.10 (s, 2H) |
| Ib-1-44 | tBuCO— | O | Me | C≡Me | Me | |
| Ib-1-45 | —CO₂CH₂CH₃ | O | Et | C≡Me | Me | δ = 1.12 (t, 3H), 1.26 (t, 3H), 1.85-1.99 (m, 1H), 2.04 (s, 3H), 2.15 (s, 3H), 2.20- |
| | | | | | | 2.25 (m, 1H), 2.42-2.50 (m, 2H), 3.35-3.45 (m, 1H), 3.79-3.89 (m, 4H), 4.11- |
| | | | | | | 4.18 (m, 3H), 4.79-4.82 (m, 1H), 4.14 (mc, 2H) |
| Ib-1-46 | tBuCO— | O | Et | C≡Me | Me | δ = 1.02 (mc, 9H), 1.87-1.95 (m, 1H), 2.03 (s, 3H), 2.20 (mc, 4H), 2.41-2.58 (m, |
| | | | | | | 3H), 3.38-3.45 (m, 1H), 3.78-3.95 (m, 4H), 4.11-4.15 (m, 1H), 4.77 (mc, 1H), |
| | | | | | | 7.10 (mc, 2H) |
| Ib-1-47 | —CO₂CH₂CH₃ | O | Et | C≡Me | Et | δ = 1.13 (dt, 6H), 1.26 (t, 3H), 1.93 (mc, 1H), 2.05 (s, 3H), 2.26 (mc, 1H), 2.40- |
| | | | | | | 2.51 (m, 4H), 3.43 (mc, 1H), 3.79-3.89 (m, 4H), 4.16 (mc, 3H), 4.86 (dd, 1H), |
| | | | | | | 7.15 (s, 2H) |
| Ib-1-48 | tBuCO— | O | Et | C≡Me | Et | δ = 1.08 (mc, 12H), 1.87 (mc, 1H), 2.05 (s, 3H), 2.16 (mc, 1H), 2.37-2.60 (m, |
| | | | | | | 5H), 3.42 (mc, 1H), 3.78-3.89 (m, 4H), 4.12 (mc, 1H), 4.83 (mc, 1H), 7.14 (s, 2H) |
| Ib-1-49 | —CO₂CH₂CH₃ | O | Me | C≡Me | OMe | δ = 1.34 (dt, 6H), 1.83-2.00 (m, 1H), 2.04 (s, 3H), 2.20 (mc, 4H), 3.32-3.44 (m, |
| | | | | | | 1H), 3.72 (s, 3H), 3.76-3.89 (m, 4H), 4.09-4.20 (m, 3H), 4.81-4.84 (m, 1H), 6.77 |
| | | | | | | (s, 1H), 6.90 (s, 1H) |
| Ib-1-50 | tBuCO— | O | Me | C≡Me | OMe | δ = 1.09 (mc, 6H), 1.79-2.00 (m, 1H), 2.05 (s, 3H), 2.16 (mc, 4H), 2.59 (q, 2H), |
| | | | | | | 3.36-3.40 (m, 1H), 3.69 (s, 3H), 3.73 (s, 3H), 3.77-3.90 (m, 4H), 4.09-4.13 (m, |
| | | | | | | 1H), 4.69-4.84 (m, 1H), 6.75 (d, 1H), 6.91 (d, 1H) |
| Ib-1-51 | —CO₂CH₂CH₃ | O | MeO | C≡Me | F | |
| Ib-1-52 | tBuCO— | O | MeO | C≡Me | F | |
| Ib-1-53 | —CO₂CH₂CH₃ | O | MeO | C≡Me | Cl | δ = 1.28 (t, 3H), 1.90-2.00 (m, 1H), 2.05 (s, 3H), 2.20-2.39 (m, 1H), 3.31-3.91 |
| | | | | | | (m, 1H), 3.76-3.90 (m 7H), 4.17-4.82 (m, 3H), 4.80.4-85 (m, 1H), 6.84 (s, 1H), |
| | | | | | | 7.08 (s, 1H) |
| Ib-1-54 | tBuCO— | O | MeO | C≡Me | Cl | |
| Ib-1-55 | —CO₂CH₂CH₃ | O | Me | OCH₂CF₃ | Me | δ = 1.25 (t, 3H), 1.93 (mc, 1H), 2.19 (d, 6H), 2.28 (mc, 1H), 3.38 (mc, 1H), |
| | | | | | | 3.79-3.92 (m, 4H), 4.13 (mc, 3H), 4.35 (q, 2H), 4.76 (mc, 1H), 6.66 (s, 2H) |
| Ib-1-56 | tBuCO— | O | Me | OCH₂CF₃ | Me | δ = 1.04 (dt, 6H), 1.85 (mc, 1H), 2.19 (mc, 7H), 2.55 (sept, 1H), 3.42 (mc, |
| | | | | | | 1H), 3.78-3.95 (m, 4H), 4.15 (mc, 1H), 4.32 (q, 2H), 4.71 (mc, 1H), 6.54 |
| | | | | | | (d, 2H) |
| Ib-1-57 | —CO₂CH₂CH₃ | O | Et | OCH₂CF₃ | Me | δ = 1.13 (dt, 3H), 1.25 (dt, 3H), 1.92 (mc, 1H), 2.18 (s, 3H), 2.24-2.29 (m, |
| | | | | | | 1H), 2.42-2.56 (m, 2H), 3.40 (mc, 1H), 3.79-3.92 (m, 4H), 4.14 (mc, 3H), |
| | | | | | | 4.33 (q, 2H), 4.81 (mc, 1H), 6.67 (s, 1H), 6.71 (s, 1H) |
| Ib-1-58 | tBuCO— | O | Et | OCH₂CF₃ | Me | δ = 1.05 (mc, 12 H), 1.85 (mc, 1H), 2.15 (mc, 1H), 2.38-2.61 (m, 5H), 3.42 |
| | | | | | | (mc, 1H), 3.78-3.95 (m, 4H), 4.115 (mc, 1H), 4.35 (q, 2H), 4.85 (mc, 1H), |
| | | | | | | 6.69 (s, 2H) |
| Ib-1-59 | —CO₂CH₂CH₃ | O | Et | OCH₂CF₃ | Et | δ = 1.11 (dt, 6H), 1.25 (t, 3H), 1.91 (mc, 1H), 2.23-2.28 (m, 1H), 2.42-2.55 |
| | | | | | | (m, 4H), 3.40 (mc, 1H), 3.79-3.91 (m, 4H), 4.18 (mc, 3H), 4.36 (q, 2H), 4.86 |
| | | | | | | (mc, 1H), 6.70 (s, 2H) |
| Ib-1-60 | tBuCO— | O | Et | OCH₂CF₃ | Et | δ = 1.10 (mc, 9H), 1.88 (mc, 1H), 2.15 (mc, 4H), 2.40-2.61 (m, 3H), 3.42 |
| | | | | | | (mc, 1H), 3.78-3.92 (m, 4H), 4.15 (mc, 1H), 4.35 (q, 2H), 4.75 (mc, 1H), |
| | | | | | | 6.54-6.59 (m, 2H) |
| Ib-1-61 | —CO₂CH₂CH₃ | O | Br | Cl | Et | |
| Ib-1-62 | tBuCO— | O | Br | Cl | Et | |
| Ib-1-63 | —CO₂CH₂CH₃ | O | MeO | Me | Me | |
| Ib-1-64 | tBuCO— | O | MeO | Me | Me | |

TABLE 5

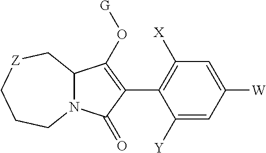

| Example | | | | | | |
|---|---|---|---|---|---|---|
| No. | Z | G | X | W | Y | ¹H-NMR (400 MHz, CDCl₃) |
| Ib-2-1 | O | CH₃CO— | Me | Me | Me | δ = 1.80 (s, 3H), 2.15 (d, 9H), 2.27 (s, 3H), 3.18 (br s, 1H), 3.55 (br s, 2H), 3.86 (mc, |
| | | | | | | 2H), 4.92 (s, 1H), 4.96 (s, 1H), 6.88 (s, 2H) |
| Ib-2-2 | O | —CO₂CH₃ | Me | Me | Me | δ = 1.81 (mc, 2H), 2.16 (s, 6H), 2.28 (s, 3H), 3.16 (mc, 1H), 3.56 (mc, 2H), 3.72 (s, |
| | | | | | | 3H), 3.87 (mc, 2H), 4.99 (s, 1H), 5.03 (s, 1H), 6.90 (s, 2H) |
| Ib-2-3 | O | —CO₂CH₂CH₃ | Me | Me | Me | δ = 1.19 (t, 3H), 1.81 (mc, 2H), 2.16 (s, 6H), 2.28 (s, 3H), 3.16 (mc, 1H), 3.55 (mc, |
| | | | | | | 2H), 3.86 (mc, 2H), 4.13 (q, 2H), 4.98 ( s, 1H), 5.04 (s, 1H), 6.89 (s, 2H) |
| Ib-2-4 | O | tBuCO— | Me | Me | Me | δ = 1.13 (s, 9H) 1.80 (mc, 2H), 2.16 (s, 6H), 2.26 (s, 3H), 3.20 (mc, 1H), 3.55 (mc, |
| | | | | | | 2H), 3.88 (mc, 2H), 4.88 (s, 1H), 4.94 (s, 1H), 6.86 (s, 2H) |
| Ib-2-5 | O | —CO₂CH₃ | Et | Me | Et | δ = 1.13 (t, 6H), 1.81 (mc, 2H), 2.33 (s, 3H), 2.46 (q, 4H), 3.15 (br s, 1H), 3.55 (mc, |
| | | | | | | 2H), 3.75 (s, 3H), 3.87 (mc, 2H), 5.00 (mc, 2H), 6.95 (s, 2H) |
| Ib-2-6 | O | —CO₂CH₂CH₃ | Et | Me | Et | δ = 1.12 (t, 6H), 1.22 (t, 3H), 1.81 (mc, 2H), 2.33 (s, 3H), 2.46 (mc, 4H), 3.18 (br s, |
| | | | | | | 1H), 3.55 (mc, 2H), 3.87 (mc, 2H), 4.16 (q, 2H), 4.99 (s, 2H), 6.95 (s, 2H) |

TABLE 6

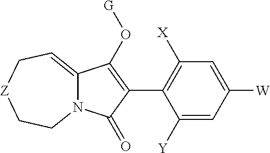

| Example | | | | | | |
|---|---|---|---|---|---|---|
| No. | Z | G | X | W | Y | ¹H-NMR (400 MHz, CDCl₃) |
| Ib-3-1 | O | —CO₂CH₂CH₃ | Me | Me | Me | δ = 1.17 (t, 3H), 2.17 (s, 6H), 2.27 (s, 3H), 3.98 (s, 4H), 4.10 (q, 2H), 4.56 (d, 2H), 5.47 (mc, |
| | | | | | | 1H), 6.88 (s, 2H), |
| Ib-3-2 | O | —CO₂CH₂CH₃ | Et | Me | Et | δ = 1.03 (t, 6H), 1.10 (t, 3H), 2.29 (s, 3H), 2.36 (mc, 4H), 3.80 (mc, 2H), 3.94 (mc, 2H), 4.11 |
| | | | | | | (q, 2H), 4.51 (d, 2H), 5.63 (mc, 1H), 6.94 (s, 2H) |
| Ib-3-3 | O | tBuCO— | Et | Me | Et | δ = 1.07 (s, 9H), 1.12 (t, 6H), 2.30 (s, 3H), 2.46 (mc, 4H), 3.99 (s, 4H), 4.56 (d, 2H), 5.27 (mc, |
| | | | | | | 1H), 6.90 (s, 2H) |
| Ib-3-4 | O | CH₃CO— | Me | Me | Et | δ = 1.13 (t, 2H), 2.10 (s, 3H), 2.16 (s, 3H), 2.30 (s, 3H), 2.48 (mc, 2H), 3.98 (s, 4H), 5.54 (d, |
| | | | | | | 2H), 5.31 (mc, 1H), 6.89 (s, 1H), 6.91 (s, 1H) |
| Ib-3-5 | O | —CO₂CH₃ | Me | Me | Et | δ = 1.13 (t, 3H), 2.17 (s, 3H), 2.30 (s, 3H), 2.47 (mc, 2H), 3.71 (s, 3H), 3.99 (s, 4H), 4.55 (d, |
| | | | | | | 2H), 5.44 (mc, 1H), 6.90 (s, 1H), 6.92 (s, 1H) |
| Ib-3-6 | 0O | —CO₂CH₂CH₃ | Me | Me | Et | δ = 1.13 (t, 3H), 1.18 (t, 3H), 2.16 (s, 3H), 2.41 (s, 3H), 2.50 (mc, 2H), 3.98 (s, 4H), 4.10 (q, |
| | | | | | | 2H), 4.56 (d, 2H), 5.45 (mc, 1H), 6.90 (s, 1H), 6.92 (s, 1H) |
| Ib-3-7 | O | tBuCO— | Me | Me | Et | δ = 1.08 (s, 9H), 1.12 (s, 3H), 2.16 (s, 3H), 2.28 (s, 3H), 2.46 (mc, 2H), 3.98 (s, 4H), 4.54 (d, |
| | | | | | | 2H), 5.27 (mc, 1H), 6.88 (s (2H) |
| Ib-3-8 | O | —CO₂CH₃ | MeO | Cl | Et | δ = 1.15 (t, 3H), 2.52 (mv, 2H), 2.73 (s, 3H), 3.75 (s, 3H), 3.97 (mc, 4H), 4.55 (d, 2H), 5.47 |
| | | | | | | (mc, 1H), 6.75 (s, 1H), 6.91 (s, 1H) |
| Ib-3-9 | O | —CO₂CH₂CH₃ | MeO | Cl | Et | δ = 1.15 (t, 3H), 1.20 (t, 3H), 2.53 (mc, 2H), 3.74 (s, 3H), 3.97 (mc, 4H), 4.12 (q, 2H), 4.55 (d, |
| | | | | | | 2H), 5.48 (mc, 1H), 6.75 (s, 1H), 6.90 (s, 1H) |
| Ib-3-10 | O | tBuCO— | MeO | Cl | Et | δ = 1.13 (s, 9H), 1.15 (s, 3H), 2.50 (mc, 2H), 3.74 (s, 3H), 3.97 (s, 4H), 5.54 (d, 2H), 5.30 (mc, |
| | | | | | | 1H), 6.72 (s, 1H), 6.88 (s, 1H) |
| Ib-3-11 | O | —CO₂CH₂CH₃ | Me | Br | Me | δ = 1.20 (t, 3H), 2.07 (s, 6H), 3.91 (mc, 4H), 4.05 (q, 2H), 4.50 (d, 2H), 5.46 (mc, 1H), 7.15 |
| | | | | | | (s, 2H) |
| Ib-3-12 | O | tBuCO— | Me | Br | Me | δ = 1.07 (s, 9H), 2.18 (s, 6H), 3.98 (s, 4H), 4.55 (d, 2H), 5.34 (mc, 1H), 7.20 (s, 2H) |
| Ib-3-13 | O | CH₃CO— | MeO | Me | Cl | δ = 2.16 (s, 3H), 2.32 (s, 3H), 3.67 (s, 3H), 3.96 (s, 4H), 4.54 (d, 2H), 5.42 (mc, 1H), 6.65 (s, |
| | | | | | | 1H), 6.87 (s, 1H) |
| Ib-3-14 | O | —CO₂CH₃ | MeO | Me | Cl | δ = 2.34 (s, 3H), 3.67 (s, 3H), 3.80 (s, 3H), 3.97 (mc, 4H), 4.54 (d, 2H), 5.51 (mc, 1H), 6.65 (s, |
| | | | | | | 1H), 6.89 (s, 1H) |
| Ib-3-15 | O | —CO₂CH₂CH₃ | MeO | Me | Cl | δ = 2.33 (t, 3H), 2.32 (s, 3H), 3.67 (s, 3H), 3.97 (mc, 4H), 4.15 (q, 2H), 4.55 (d, 2H) 5.53 (mc, |
| | | | | | | 1H), 6.65 (s, 1H), 6.88 (s, 1H) |
| Ib-3-16 | O | tBuCO— | MeO | Me | Cl | δ = 1.18 (s, 9H), 3.32 (s, 3H), 3.77 (s, 3H), 3.96 (s, 4H), 4.54 (d, 2H), 5.35 (mc, 1H), 6.12 (s, |
| | | | | | | 1H), 6.85 (s, 1H) |

### Preparation of the precursors II

### 4-[(2,6-Diethyl-4-methylphenyl)acetyl]-1,4-oxazepan-5-one

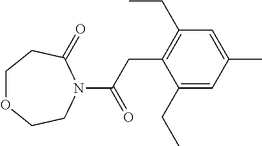 [SMILES: CCc1cc(C)cc(CC)c1CC(=O)N1CCOCCC1=O]

### 4.0 g (34.7 mol) of 1,4-oxazepan-5-one (CAS: 10341-26-1) are dissolved in 150 ml of THF, and 1.0 equivalents of n-butyllithium are added dropwise at below −70° C. The mixture is stirred for another 20 min, and a solution of 7.2 g (31.9 mmol) of [2,6-diethyl-4-methylphenyl]acetyl chloride is then added dropwise at this temperature over 20 min and stirring is continued at this temperature for 20 min and at room temperature for a further hour. For work-up, 100 ml of 5% strength sodium bicarbonate solution and 500 ml of diethyl ether are added, the organic phase is separated off and the aqueous phase is extracted twice with in each case 200 ml of diethyl ether. The combined organic phases are washed with 100 ml of sat. sodium chloride solution, dried over sodium sulfate and concentrated. Purification of the residue by column chromatography gives 8.30 g of the target product as a crystalline solid of m.p. 74° C.

### 4-[(2,6-Diethyl-4-methylphenyl)acetyl]-2,3,4,7-tetrahydro-1,4-oxazepin-5-yl diphenyl phosphate

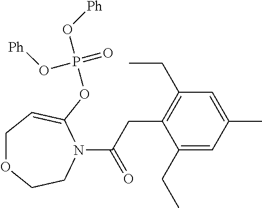 [SMILES: CCc1cc(C)cc(CC)c1CC(=O)N1CCOCC=C1OP(=O)(Oc1ccccc1)Oc1ccccc1]

8.2 g (27.0 mmol) of the above compound are initially charged in 250 ml of THF, and 6.47 g (1.2 eq) of potassium hexamethyldisilazide in 50 ml of THF are added dropwise at below −70° C. over an hour. The mixture is stirred for another 90 min, and a solution of 8.71 g (32.4 mmol) of diphenyl chlorophosphate in 50 ml of THF is added dropwise at below −70° C. After 60 min at this temperature, the mixture is allowed to warm to room temperature, stirred with 93 ml of 5% strength sodium hydroxide solution and extracted three times with in each case 100 ml of diethyl ether and once with sat. sodium chloride solution. After drying over sodium sulfate and distillative removal of the solvent, the semicrystalline residue obtained is purified by chromatography on silica gel. This gives 10.6 g of the target product in the form of colourless crystals of m.p. 110-112° C.

### Methyl 4-[(2,6-diethyl-4-methylphenypacetyl]-2,3,4,5-tetrahydro-1,4-oxazepine-5-carboxylate

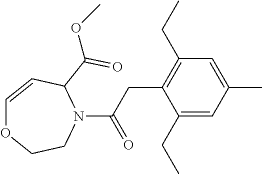 [SMILES: CCc1cc(C)cc(CC)c1CC(=O)N1CCOC=CC1C(=O)OC]

10.5 g (19.6 mmol) of 4-[(2,6-diethyl-4-methylphenyl)acetyl]-2,3,4,7-tetrahydro-1,4-oxazepin-5-yl diphenyl phosphate are dissolved in 200 ml of DMF, degassed under reduced pressure and covered with argon. 0.44 g (0.1 eq) of palladium acetate and 1.0 g (0.2 eq) of triphenylphosphine are added and the mixture is covered with carbon monoxide and stirred at room temperature for another 30 min, during which time the colour of the solution changes to black. The mixture is then briefly degassed, 4.0 g (39.2 mmol) of triethylamine and 25.8 g (0.8 mol) of methanol are added and the mixture is once more covered with carbon monoxide and stirred at 45° C. for 60 min. The reaction solution is then poured into ice water and the aqueous phase is extracted three times with in each case 300 ml of diethyl ether, washed with 100 ml of sat. sodium chloride solution, dried over sodium sulfate and concentrated. This gives 2.6 g of a light-yellow oil which is dissolved in 40 ml of THF and, at -70° C. and over a period of one hour, added dropwise to a solution of 0.38 g (2.7 mmol) of potassium tert-butoxide in 20 ml of THF. Stirring at this temperature is continued for another 10 min, 10 ml of a 1 molar buffer solution (pH=4.65) are then added, the mixture is allowed to warm to room temperature and about 20 ml of water are added. The aqueous phase is extracted twice with in each case 50 ml of diethyl ether, washed with 20 ml of sat. sodium chloride solution, dried over sodium sulfate and concentrated. Purification of the residue by column chromatography gives 0.32 g of the target product in the form of an oil with a yellow hue.

### Methyl 4-[(2,6-diethyl-4-methylphenypacetyl]-1,4-oxazepane-5-carboxylate

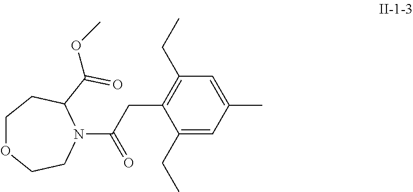 [SMILES: CCc1cc(C)cc(CC)c1CC(=O)N1CCOCCC1C(=O)OC]

0.50 g (1.4 mmol) of the precursor in 50 ml of methanol is, after addition of palladium/carbon (5%), hydrogenated at room temperature and standard pressure for about 90 min. Filtration and distillative removal of the solvent gives, without further purification, the target compound as a colourless oil in almost quantitative yield.

### Benzyl 5-oxo-1,4-oxazepane-4-carboxylate

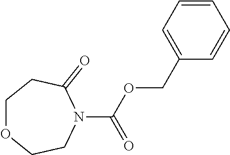 [SMILES: O=C1CCOCCN1C(=O)OCc1ccccc1]

5.0 g (43.4 mol) of 1,4-oxazepan-5-one (CAS: 10341-26-1) are dissolved in 100 ml of THF, and 1.1 equivalents of n-butyllithium are added dropwise at below −70° C. The mixture is stirred for another 30 min, 7.8 g (46.0 mmol) of benzyloxycarbonyl chloride are added dropwise and the mixture is stirred at −70° C. for another 1 h and then warmed to RT. For work-up, 100 ml of saturated NH₄Cl solution are added and the mixture is extracted three times with ethyl acetate. The combined organic phases are washed with 100 ml of sat. sodium chloride solution, dried over sodium sulfate and concentrated. Purification of the residue by column chromatography gives 10.0 g of the target product as a colourless oil.

### Benzyl 5-diphenoxyphosphoryloxy-3,7-dihydro-2H-1,4-oxazepine-4-carboxylate

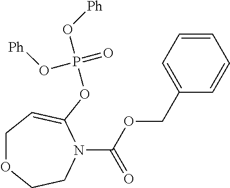 [SMILES: O=C(OCc1ccccc1)N1CCOCC=C1OP(=O)(Oc1ccccc1)Oc1ccccc1]

10.0 g (40.1 mmol) of the above compound are initially charged in 370 ml of THF, and 9.604 g (1.2 eq) of potassium hexamethyldisilazide in 50 ml of THF are added dropwise at below −70° C. over an hour. The mixture is stirred for another 90 min, and a solution of 12.9 g (48.1 mmol) of diphenyl chlorophosphate in 75 ml of THF is added dropwise at below −70° C. After 60 min at this temperature, the mixture is allowed to warm to room temperature, stirred with 130 ml of 5% strength sodium hydroxide solution, the volatile components are removed and the residue is extracted three times with in each case 100 ml of diethyl ether and once with sat. sodium chloride solution. After drying over sodium sulfate and distillative removal of the solvent, the residue obtained is purified by chromatography on silica gel. This gives 15.9 g of the target product as a slightly yellow oil.

### O4-Benzyl O5-methyl 3,5-dihydro-2H-1,4-oxazepine-4,5-dicarboxylate

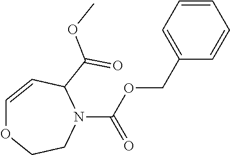 [SMILES: COC(=O)C1C=COCCN1C(=O)OCc1ccccc1]

15.9 g (33.0 mmol) of the above precursor are dissolved in 340 ml of DMF, and the solution is degassed under reduced pressure and covered with argon. 0.74 g (0.1 eq) of palladium acetate and 1.7 g (0.2 eq) of triphenylphosphine are added and the mixture is covered with carbon monoxide and stirred at room temperature for another 30 min, during which time the colour of the solution changes to black. The mixture is then briefly degassed, 6.7 g (66.1 mmol) of triethylamine and 42.4 g (1.3 mol) of methanol are added and the mixture is once more covered with carbon monoxide and stirred at 45° C. for 60 min. The reaction solution is then poured into ice water and the aqueous phase is extracted three times with in each case 300 ml of diethyl ether, washed with 100 ml of sat. sodium chloride solution, dried over sodium sulfate and concentrated. Chromatography on silica gel gives 6.0 g of the target compound in the form of a yellow oil.

### Methyl 1,4-oxazepane-5-carboxylate

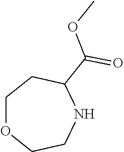

12.3 g (42 2 mmol) of the precursor in 150 ml of methanol are, after addition of palladium/carbon (10%), hydrogenated at room temperature and standard pressure for about 3 h. Filtration and distillative removal of the solvent gives, without further purification, the target compound as a colourless oil in almost quantitative yield. δ (400 MHz, CDCl₃)=1.82-1.87 (m, 1H), 2.08-2.29 (m, 1H), 2.59-2.64 (m, 1H), 2.90-2.94 (m 1H), 3.49-3.58 (m, 3H), 3.62 (s, 3H), 3. 68 (t, 2H)

### Methyl 4-[2-(2-bromo-6-ethyl-4-methylphenyl) acetyl]-1,4-oxazepane-5-carboxylate

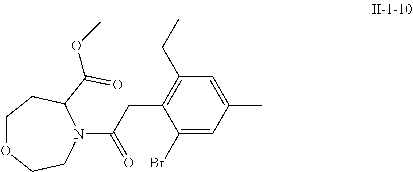 [SMILES: CCc1cc(C)cc(Br)c1CC(=O)N1CCOCCC1C(=O)OC]

1.1 g (4.1 mmol) of 2-(2-bromo-6-ethyl-4-methylphenyl)acetic acid are dissolved in 20 ml of dichloromethane and, after addition of a few drops of dimethylformamide, 1.1 g (2.0 eq.) of oxalyl chloride are added dropwise. After the evolution of gas has ceased, the volatile components are removed completely under reduced pressure, the residue is taken up again in 20 ml of dichloromethane and the mixture is, at 0° C., added dropwise to a suspension of 0.65 g (4.1 mmol) of methyl 1,4-oxazepane-5-carboxylate and 1.70 g (4.0 eq) of triethylamine in 20 ml of dichloromethane. After 3 h at RT, water is added, the phases are separated and the organic phase is concentrated. Chromatography on silica gel gives 1.05 g of the target compound as a colourless oil.

The following precursors according to the invention were prepared analogously to the preparation of precursors II-1-3 and II-1-10:

TABLE 7

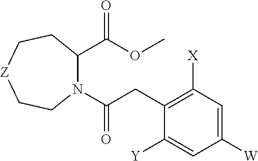

| Example | | | | | |
|---|---|---|---|---|---|
| No. | Z | X | W | Y | ¹H-NMR (400 MHz, CDCl₃) |
| II-1-1 | O | Me | Me | Me | δ = 2.22 (s, 6H), 2.36 (s, 3H), 3.69 (s, 3H), 5.00 (mc, 1H), 6.85 |
| | | | | | (s, 2H) |
| II-1-2 | O | Me | Me | Et | δ = 1.16 (t, 3H), 2.18 (s, 3H), 2.21 (s, 3H), 2.54 (q, 2H), 3.69 (s, |
| | | | | | 3H), 5.00 (mc, 1H), 6.86 (s, 2H) |
| II-1-3 | O | Et | Me | Et | δ = 1.17 (t, 6H), 2.29 (s, 3H), 2.53 (mc, 4H), 3.68 (s, 3H), 6.88 |
| | | | | | (s, 2H) |
| II-1-4 | O | Et | H | Me | δ = 1.18 (t, 3H), 2.22 (s, 3H), 2.58 (q, 2H), 3.70 (s, 3H), 5.00 |
| | | | | | (mc, 1H), 7.10 (mc, 3H) |
| II-1-5 | O | Cl | Me | MeO | δ = 2.37 (s, 3H), 3.69 (s, 3H) 3.74 (s, 3H), 5.05 (mc, 1H), 6.84 |
| | | | | | (s, 1H), 6.59 (s, 1H) |
| II-1-6 | O | Me | Br | Me | δ = 2.20 (s, 6H), 3.70 (s, 3H), 5.01 (mc, 1H), 7.18 (s, 2H) |
| II-1-7 | O | Me | Me | Cl | |
| II-1-8 | O | Et | Me | Cl | |
| II-1-9 | O | Me | Me | Br | |
| II-1-10 | O | Et | Me | Br | δ = 1.15 (t, 3H), 2.02 (s, 3H), 3.21 (s, 3H), 2.63 (q, 2H), 3.72 (s, |
| | | | | | 3H), 5.01 (mc, 1H), 7.08 (s, 1H), 7.10 (s, 1H) |
| II-1-11 | O | Et | Cl | EtO | δ = 1.18 (t, 3H), 1.40 (t, 3H), 2.60 (q, 2H), 3.71 (s, 3H), 3.95- |
| | | | | | 4.03 (m, 4H), 4.98 (mc, 1H), 6.69 (s, 1H), 6.82 (s, 1H) |
| II-1-12 | O | Me | C≡Me | Me | δ = 2.02 (s, 3H), 2.22 (s, 6H), 3.76 (s, 3H), 5.00 (mc, 1H), 7.07 |
| | | | | | (s, 2H) |
| II-1-13 | O | Et | C≡Me | Me | δ = 1.18 (t, 3H), 2.28 (s, 3H), 2.59 (q, 2H), 3.72 (s, 3H), 3.89 (s, |
| | | | | | 2H), 5.01 (mc, 1H), 6.96 (s, 2H) |
| II-1-14 | O | Et | C≡Me | Et | |
| II-1-15 | O | Me | C≡Me | MeO | δ = 2.03 (s, 3H), 2.26 (s, 3H), 3.77 (s, 3H), 4.98 (mc, 1H), 6.75 |
| | | | | | (s, 1H), 6.87 (s, 1H) |
| II-1-16 | O | MeO | C≡Me | F | |
| II-1-17 | O | MeO | C≡Me | Cl | δ = 2.04 (s, 3H), 3.71 (s, 3H), 3.81 (s, 3H), 5.04 (mc, 1H), 6.80 |
| | | | | | (s, 1H), 7.05 (s, 1H) |

TABLE 8

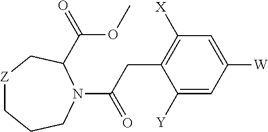

| Example | | | | | |
|---|---|---|---|---|---|
| No. | Z | X | W | Y | ¹H-NMR (400 MHz, CDCl₃) |
| II-2-1 | O | Me | Me | Me | δ = 2.22 (3H), 2.28 (s, 6H), 3.77 (s, 3H), 5.16 (mc, 1H), 6.84 (s, 2H) |
| II-2-2 | O | Me | Et | Et | δ = 1.18 (t, 6H), 2.28 (s, 3H), 2.54 (q, 4H), 3.79 (s, 3H), 5.16 (mc, 1H), 6.87 |
| | | | | | (s, 2H) |

TABLE 9

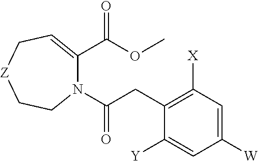

| Example | | | | | |
|---|---|---|---|---|---|
| No. | Z | X | W | Y | ¹H-NMR (400 MHz, CDCl₃) |
| II-3-1 | O | Me | Me | Me | δ = 2.20 (s, 3H), 2.24 (s, 6H), 3.47 (br s, 1H), 3.73 (mc, 1H), 3.83 (mc, 2H) |
| | | | | | 3.86 (s, 3H), 4.30 (br s, 2H), 6.84 (s, 2H), 6.95 (mc, 1H) |
| II-3-2 | O | Me | Me | Et | δ = 1.16 (t, 3H), 1.72 (s, 2H), 2.19 (s, 3H), 2.26 (s, 3H), 2.54 (q, 2H), 3.70- |
| | | | | | 3.83 (m, 2H), 2.88 (s, 3H), 4.29 (mc, 2H), 6.85 (s, 2H), 6.96 (mc, 1H) |
| II-3-3 | O | Et | Me | Et | δ = 1.17 (t, 6H), 2.28 (s, 3H), 2.53 (q, 4H), 3.51 (s, 2H), 3.69-3.87 (m, 2H), |
| | | | | | 3.88 (s, 3H), 4.29 (mc, 2H), 6.87 (s, 2H), 6.97 (mc, 1H) |
| II-3-4 | O | Et | Cl | MeO | δ = 1.17 (s, 3H), 2.58 (q, 2H), 3.49-3.86 (m, 4H), 3.77 (s, 3H), 3.88 (s, 3H), |
| | | | | | 4.25 (mc, 2H), 6.69 (s, 1H), 6.83 (s, 1H), 6.98 (mc, 1H) |
| II-3-5 | O | Cl | Me | MeO | δ = 2.30 (s, 3H), 3.67-3.79 (m, 4H), 3.80 (s, 3H), 3.90 (s, 3H), 4.27 (mc, 2H), |
| | | | | | 6.58 (s, 1H), 6.82 (s, 1H), 7.00 (mc, 1H) |
| II-3-6 | O | Me | Br | Me | δ = 2.21 (s, 6H), 3.46 (br s, 2H), 3.72-3.83 (m, 2H), 3.88 (s, 3H), 4.31 (br s, |
| | | | | | 2H), 6.96 (mc, 1H), 7.17 (s, 2H) |

### B. FORMULATION EXAMPLES

a) A dusting product is obtained by mixing 10 parts by weight of a compound of the formula (I) and/or salts thereof and 90 parts by weight of talc as inert substance and comminuting the mixture in an impact mill.

b) A readily water-dispersible, wettable powder is obtained by mixing 25 parts by weight of a compound of the formula (I) and/or salts thereof, 64 parts by weight of kaolin-containing quartz as inert substance, 10 parts by weight of potassium lignosulfonate and 1 part by weight of sodium oleoylmethyltaurate as wetting agent and dispersant and grinding in a pinned-disc mill.

c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range e.g. about 255 to more than 277° C.) and grinding to a fineness of below 5 microns in an attrition ball mill.

d) An emulsifiable concentrate is obtained from 15 parts by weight of a compound of the formula (I) and/or salts thereof, 75 parts by weight of cyclohexanone as solvent and 10 parts by weight of oxethylated nonylphenol as emulsifier.

e) Water-dispersible granules are obtained by mixing

75 parts by weight of a compound of the formula (I) and/or salts thereof,

10 parts by weight of calcium lignosulfonate,

5 parts by weight of sodium laurylsulfate,

3 parts by weight of polyvinyl alcohol and

7 parts by weight of kaolin,

grinding the mixture in a pinned-disc mill, and granulating the powder in a fluidized bed by spray application of water as a granulating liquid.

f) Water-dispersible granules are also obtained by homogenizing and precomminuting, in a colloid mill,

25 parts by weight of a compound of the formula (I) and/or salts thereof,

5 parts by weight of sodium 2,2′dinaphthylmethane-6,6′disulfonate,

2 parts by weight of sodium oleoylmethyltaurate,

1 part by weight of polyvinyl alcohol,

17 parts by weight of calcium carbonate and

50 parts by weight of water,

then grinding the mixture in a bead mill and atomizing and drying the resulting suspension in a spray tower by means of a one-phase nozzle.

### C. BIOLOGICAL DATA

1. Pre-Emergence Herbicidal Effect and Crop Plant Compatibility

Seeds of monocotyledonous and dicotyledonous weed plants and crop plants are laid out in sandy loam soil in wood-fibre pots and covered with soil. The compounds of the invention, formulated in the form of wettable powders (WP) or as emulsion concentrates (EC), are then applied to the surface of the covering soil as aqueous suspension or emulsion at a water application rate equating to 600 to 800 L/ha with addition of 0.2% wetting agent.

After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants).

Undesired plants/weeds:

ALOMY: Alopecurus myosuroides

AVEFA: Avena fatua

CYPES: Cyperus esculentus

ECHCG: Echinochloa crus-galli

LOLMU: Lolium multiflorum

SETVI: Setaria viridis

TABLE 10
Pre-emergence action

| | Dosage | | | | | |
|---|---|---|---|---|---|---|
| Ex. No. | [g/ha] | ALOMY | AVEFA | ECHCG | LOLMU | SETVI |
| Ia-1-1 | 320 | 100 | 100 | 100 | 100 | 90 |
| Ia-1-2 | 320 | 100 | 100 | 100 | 100 | 100 |
| Ia-1-3 | 320 | 100 | 100 | 100 | 100 | 100 |
| Ia-1-4 | 320 | 80 | 100 | 100 | 100 | 100 |
| Ia-1-6 | 320 | 100 | 100 | 100 | 100 | 100 |
| Ia-1-8 | 320 | 90 | | | 100 | |
| Ia-2-1 | 320 | 80 | | | 100 | |
| Ia-2-2 | 320 | 80 | 90 | 100 | 100 | 100 |
| Ia-3-3 | 320 | 80 | | | 100 | |
| Ia-3-4 | 320 | 80 | 80 | 90 | 90 | 100 |
| Ib-1-1 | 320 | 100 | 100 | 100 | 100 | 100 |
| Ib-1-2 | 320 | 100 | 100 | 100 | 100 | 100 |
| Ib-1-3 | 320 | 100 | 100 | 100 | 100 | 100 |
| Ib-1-4 | 320 | 100 | 100 | 100 | 100 | 100 |
| Ib-1-5 | 320 | 100 | 100 | 100 | 100 | 100 |
| Ib-1-6 | 320 | 100 | 100 | 100 | 100 | 100 |
| Ib-1-7 | 320 | 100 | 100 | 100 | 100 | 100 |
| Ib-1-8 | 320 | 100 | 100 | 100 | 100 | 100 |
| Ib-1-9 | 320 | 100 | 100 | 100 | 100 | 100 |
| Ib-1-10 | 320 | 80 | 90 | 100 | 100 | 100 |
| Ib-1-11 | 320 | 90 | 90 | 100 | 100 | 100 |
| Ib-1-12 | 320 | 100 | 90 | 100 | 100 | 100 |
| Ib-1-13 | 320 | 80 | 90 | 100 | 100 | 100 |
| Ib-1-14 | 320 | 80 | 90 | 100 | 100 | 100 |
| Ib-1-15 | 320 | 100 | | 100 | 100 | 100 |
| Ib-1-16 | 320 | 100 | 100 | 100 | 100 | 100 |
| Ib-1-17 | 320 | 100 | 100 | 100 | | 100 |
| Ib-1-18 | 320 | 100 | 100 | 100 | 100 | 100 |
| Ib-1-21 | 320 | 100 | 90 | 100 | 100 | 100 |
| Ib-1-22 | 320 | 90 | 100 | 100 | 100 | 100 |
| Ib-1-23 | 320 | 100 | 90 | 100 | 100 | 100 |
| Ib-1-24 | 320 | 90 | 90 | 100 | 100 | 100 |
| Ib-1-26 | 320 | 90 | | | 90 | |
| Ib-3-5 | 320 | 100 | | | 80 | |
| Ib-3-10 | 320 | 90 | | 100 | 100 | 100 |
| Ib-3-13 | 320 | 100 | 90 | 100 | 100 | 100 |
| Ib-3-16 | 320 | 90 | | 80 | 90 | 90 |

As the results from Table 10 show, compounds according to the invention have a good herbicidal pre-emergence effectiveness against a broad spectrum of weed grasses and weeds. For example, the compounds No. Ia-1-1, Ia-1-2, Ia-1-4, Ib-1-1, Ib-1-2 and Ib-1-9, at an application rate of 320 g/ha, in each case exhibit an 80-100% effect against Alopecurus myosuroides, Avena fatua, Cyperus esculentus, Echinochloa crus-galli, Lolium multiflorum and Setaria viridis. The compounds of the invention are therefore suitable for control of unwanted plant growth by the pre-emergence method.

2. Post-Emergence Herbicidal Effect and Crop Plant Compatibility

Seeds of monocotyledonous and dicotyledonous weed and crop plants are laid out in sandy loam soil in wood-fibre pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants as aqueous suspension or emulsion at a water application rate equating to 600 to 800 L/ha with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants).

TABLE 11
Post-emergence effectiveness

| | Dosage | | | | | |
|---|---|---|---|---|---|---|
| Ex. No. | [g/ha] | ALOMY | AVEFA | ECHCG | LOLMU | SETVI |
| Ia-1-1 | 320 | 100 | 100 | 100 | 100 | 100 |
| Ia-1-2 | 320 | 100 | 100 | 100 | 100 | 100 |
| Ia-1-3 | 320 | 100 | 100 | 100 | 100 | 100 |
| Ia-1-4 | 320 | 100 | 100 | 100 | 100 | 100 |
| Ia-1-6 | 320 | 100 | 100 | 100 | 100 | 100 |
| Ia-2-1 | 320 | 100 | 100 | 100 | 100 | 100 |
| Ia-2-2 | 320 | 100 | 100 | 100 | 100 | 100 |
| Ia-3-1 | 320 | | 80 | 90 | | |
| Ia-3-2 | 320 | | 100 | 100 | | |
| Ia-3-3 | 320 | | | 100 | | 100 |
| Ia-3-4 | 320 | 100 | 100 | 100 | 100 | 100 |
| Ib-1-1 | 320 | 100 | 100 | 100 | 100 | 100 |
| Ib-1-2 | 320 | 100 | 100 | 100 | 100 | 100 |
| Ib-1-3 | 320 | 100 | 100 | 100 | 100 | 100 |
| Ib-1-4 | 320 | 100 | 100 | 100 | 100 | 100 |
| Ib-1-6 | 320 | 100 | 100 | 100 | 100 | 100 |
| Ib-1-7 | 320 | 100 | 100 | 100 | 100 | 100 |
| Ib-1-8 | 320 | 100 | 100 | 100 | 100 | 100 |
| Ib-1-9 | 320 | 100 | 100 | 100 | 100 | 100 |
| Ib-1-10 | 320 | 100 | 100 | 100 | 100 | 100 |
| Ib-1-11 | 320 | 100 | 100 | 100 | 100 | 100 |
| Ib-1-12 | 320 | 100 | 100 | 100 | 100 | 100 |
| Ib-1-13 | 320 | 100 | 90 | 100 | 100 | 100 |
| Ib-1-14 | 320 | 90 | 100 | 100 | 100 | 100 |
| Ib-1-15 | 320 | 100 | 100 | 100 | 100 | 100 |
| Ib-1-16 | 320 | 100 | 100 | 100 | 100 | 100 |
| Ib-1-17 | 320 | 100 | 100 | 100 | 100 | 100 |
| Ib-1-18 | 320 | 100 | 100 | 100 | 100 | 100 |
| Ib-2-1 | 320 | | | 100 | | 100 |
| Ib-2-2 | 320 | | | 90 | | 90 |
| Ib-2-3 | 320 | | | 90 | | 90 |
| Ib-2-4 | 320 | | | 90 | | 100 |
| Ib-2-5 | 320 | | | 100 | | 100 |
| Ib-2-6 | 320 | | | 100 | | 100 |
| Ib-3-2 | 320 | | | 80 | | 90 |
| Ib-3-3 | 320 | | | 90 | | 100 |
| Ib-3-4 | 320 | 90 | 80 | 90 | 80 | 100 |
| Ib-3-5 | 320 | | | 90 | | 90 |
| Ib-3-6 | 320 | | | 90 | | 90 |
| Ib-3-7 | 320 | | | 90 | | 100 |
| Ib-3-9 | 320 | | | 90 | | 100 |
| Ib-3-10 | 320 | 100 | 100 | 100 | 100 | 100 |
| Ib-3-11 | 320 | | | 80 | | 90 |

As the results from Table 11 show, compounds according to the invention have a good herbicidal post-emergence effectiveness against a broad spectrum of weed grasses and weeds. For example, the compounds No. Ia-2-1, Ia-2-2, Ib-1-1, Ib-1-2, Ib-1-3, Ib-1-4, Ib-1-6 and Ib-1-7, at an application rate of 320 g/ha, in each case exhibit a 90-100% effect against Alopecurus myosuroides, Avena fatua, Echinochloa crus-galli, Lolium multiflorum and Setaria viridis. The compounds of the invention are therefore suitable for control of unwanted plant growth by the post-emergence method.

1. Compound of formula (I) 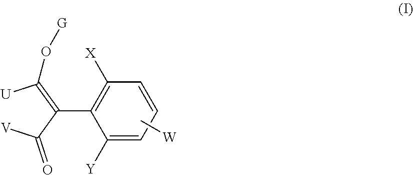
or an agrochemically acceptable salt thereof in which
X represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy or halogen;
Y represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy or halogen;
W represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy, C₂-C₆-alkenyl, C₂-C₆-alkynyl, halogen or phenyl which is unsubstituted or may optionally be substituted by one or more substituents independently of one another selected from the group consisting of C₁-C₃-alkyl, C₁-C₄-haloalkyl, C₃-C₆-cycloalkyl, hydroxy, C₁-C₆-alkoxy, C₁-C₆-haloalkoxy and halogen;
U and V in each case together form a seven-membered ring of the T¹-T⁴ type, 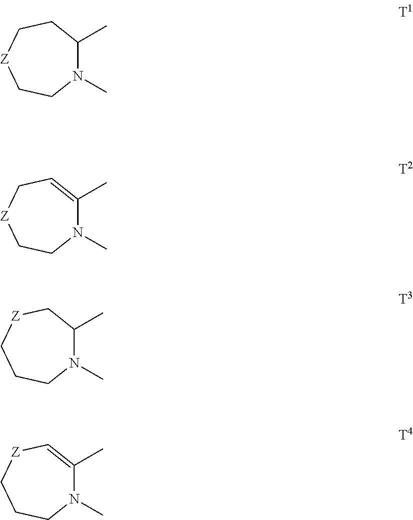
where Z represents an oxygen atom, a group —S(O)ₙ— or a group —N(OR¹)—;
n represents 0, 1 or 2;
R¹ represents hydrogen, C₁-C₄-alkyl, C₁-C₄-haloalkyl or C₁-C₄-alkanoyl;
G represents hydrogen, a removable group L or a cation E, where L represents one of the radicals below 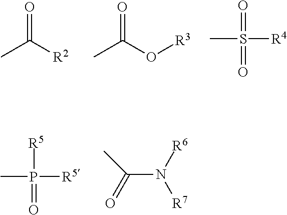
R² represents C₁-C₄-alkyl or C₁-C₃-alkoxy-C₁-C₄-alkyl;
R³ represents C₁-C₄-alkyl;
R⁴ represents C₁-C₄-alkyl or phenyl which is unsubstituted or may optionally be substituted by one or more substituents independently of one another selected from the group consisting of halogen, C₁-C₄-alkyl, C₁-C₄-haloalkyl, C₁-C₄-alkoxy, C₁-C₄-haloalkoxy, nitro or cyano;
R⁵ and R^{5′} each independently of one another represent methoxy or ethoxy;
R⁶ and R⁷ each independently of one another represent methyl, ethyl or phenyl or together form a saturated 5-, 6- or 7-membered ring or together form a saturated 5-, 6- or 7-membered heterocycle having an oxygen or sulfur atom;
E represents an alkali metal ion, an ion equivalent of an alkaline earth metal, an ion equivalent of aluminium, an ion equivalent of a transition metal, a magnesium halogen cation or an ammonium ion, in which optionally one, two, three or all four hydrogen atoms are replaced by identical or different radicals from the groups C₁-C₅-alkyl, C₁-C₆-alkoxy or C₃-C₇-cycloalkyl, which may in each case be substituted one or more times with fluorine, chlorine, bromine, cyano, hydroxy or be interrupted by one or more oxygen or sulfur atoms, or a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO) or 1,5-diazabicyclo[4.3.0]undec-7-ene (DBU), or a heterocyclic ammonium cation, for example in each case protonated pyridine, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, pyrrole, imidazole, quinoline, quinoxaline, 1,2-dimethylimidazole, 1,3-dimethylimidazolium methyl sulfate, or a sulfonium ion.

2. Compound of formula (I) or an agrochemically acceptable salt thereof according to claim 1, wherein X represents hydrogen, C₁-C₄-alkyl, C₁-C₄-haloalkyl, C₁-C₆-alkoxy, C₁-C₃-alkoxy-C₁-C₄-alkyl, C₁-C₃-haloalkoxy or halogen.

3. Compound of formula (I) or an agrochemically acceptable salt thereof according to claim 1, wherein Y represents C₁-C₄-alkyl, C₁-C₄-haloalkyl, C₁-C₃-alkoxy-C₁-C₄-alkyl, C₁-C₃-haloalkoxy or halogen.

4. Compound of formula (I) or an agrochemically acceptable salt thereof according to claim 1, wherein W represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy, C₂-C₆-alkenyl, C₂-C₆-alkynyl, halogen or phenyl which is unsubstituted or may optionally be substituted by one or more substituents independently of one another selected from the group consisting of C₁-C₃-alkyl, C₁-C₄-haloalkyl, C₃-C₆-cycloalkyl, C₁-C₆-alkoxy, C₁-C₆-haloalkoxy and halogen.

5. Compound of formula (I) or an agrochemically acceptable salt thereof according to claim 1, wherein U and V in each case together form a seven-membered ring of the T¹-T⁴ type, 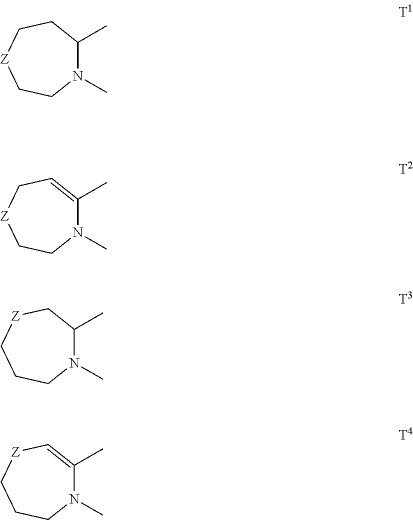
where Z represents an oxygen atom, a group —S(O)ₙ— or a group —N(OR¹)—,
n represents 0, 1 or 2, and
R¹ represents C₁-C₄-alkyl, C₁-C₄-haloalkyl or C₁-C₄-alkanoyl.

6. Compound of formula (I) or an agrochemically acdeptable salt thereof according to claim 1, wherein G represents hydrogen, a removable group L or a cation E, where L represetns one of the radicals below 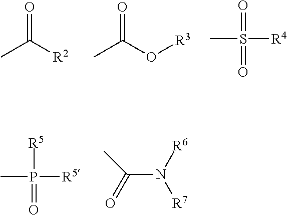
R² represents C₁-C₄-alkyl or C₁-C₃-alkoxy-C₁-C₄-alkyl,
R³ represents C₁-C₄-alkyl,
R⁴ represents C₁-C₄-alkyl or phenyl which is unsubstituted or may optionally be substituted by one or more substituents independently of one another selected from the group consisting of halogen and C₁-C₄-alkyl,
R⁵ and R^{5′} represent methoxy or ethoxy,
R⁶ and R⁷ each independently of one another represent methyl, ethyl or phenyl, and
E represents an alkali metal ion, an ion equivalent of an alkaline earth metal, an ion equivalent of aluminium, an ion equivalent of a transition metal, a magnesium halogen cation or an ammonium ion, in which optionally one, two, three or all four hydrogen atoms are replaced by identical or different radicals from the groups C₁-C₅-alkyl, C₁-C₆-alkoxy or C₃-C₇-cycloalkyl, or a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO), 1,5-diazabicyclo[4.3.0]undec-7-ene (DBU) or choline.

7. Compound of formula (I) or an agrochemically acceptable salt thereof according to claim 1, wherein
X represents hydrogen, C₁-C₄-alkyl, C₁-C₄-haloalkyl, C₁-C₆-alkoxy, C₁-C₃-alkoxy-C₁-C₄-alkyl, C₁-C₃-haloalkoxy or halogen,
Y represents C₁-C₄-alkyl, C₁-C₄-haloalkyl, C₁-C₃-alkoxy-C₁-C₄-alkyl, C₁-C₃-haloalkoxy or halogen,
W represents hydrogen, C₁-C₄-alkyl, C₁-C₄-haloalkyl, C₃-C₆-cycloalkyl, C₁-C₆-alkoxy, C₁-C₃-alkoxy-C₁-C₄-alkyl, C₁-C₆-haloalkoxy, C₂-C₆-alkenyl, C₂-C₆-alkynyl, halogen or phenyl which is unsubstituted or may optionally be substituted by one or more substituents independently of one another selected from the group consisting of C₁-C₃-alkyl, C₁-C₄-haloalkyl, C₃-C₆-cycloalkyl, C₁-C₆-alkoxy, C₁-C₆-haloalkoxy and halogen,
U and V in each case together form a seven-membered ring of the T¹-T⁴ type, 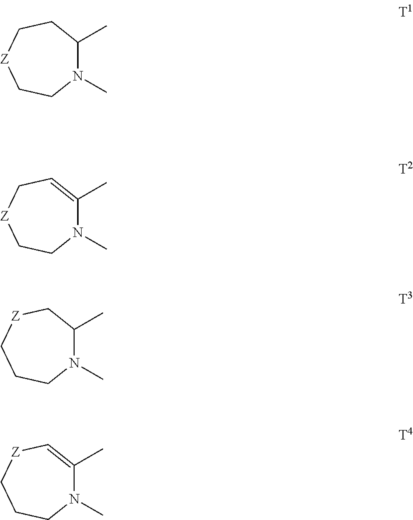
where Z represents an oxygen atom, a group —S(O)ₙ— or a group —N(OR¹)—,
n is 0, 1 or 2,
R¹ represents C₁-C₄-alkyl, C₁-C₄-haloalkyl or C₁-C₄-alkanoyl,
G represents hydrogen, a removable group L or a cation E, where L represents one of the radicals below: 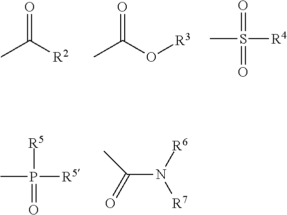
R² represents C₁-C₄-alkyl or C₁-C₃-alkoxy-C₁-C₄-alkyl,
R³ represents C₁-C₄-alkyl,
R⁴ represents C₁-C₄-alkyl or phenyl which is unsubstituted or may optionally be substituted by one or more substituents independently of one another selected from the group consisting of halogen and C₁-C₄-alkyl,
R⁵ and R^{5′} represent methoxy or ethoxy,
R⁶ and R⁷ each independently of one another represent methyl, ethyl or phenyl,
E represents an alkali metal ion, an ion equivalent of an alkaline earth metal, an ion equivalent of aluminium, an ion equivalent of a transition metal, a magnesium halogen cation or an ammonium ion, in which optionally one, two, three or all four hydrogen atoms are replaced by identical or different radicals from the groups C₁-C₅-alkyl, C₁-C₆-alkoxy or C₃-C₇-cycloalkyl, or a cyclic secondary or tertiary aliphatic or heteroaliphatic ammonium ion, for example morpholinium, thiomorpholinium, piperidinium, pyrrolidinium, or in each case protonated 1,4-diazabicyclo[2.2.2]octane (DABCO), 1,5-diazabicyclo[4.3.0]undec-7-ene (DBU) or choline.

8. Herbicidal composition comprising a compound of formula (I) according to claim 1 or an agrochemically acceptable salt thereof, and optionally an agrochemically acceptable carrier, diluent and/or solvent.

9. Herbicidal composition according to claim 8, comprising at least one further pesticidally active substance from the group of insecticides, acaricides, herbicides, fungicides, safeners and growth regulators.

10. Herbicidal composition according to claim 8, comprising a safener.

11. Herbicidal composition according to claim 8, comprising a further herbicide.

12. Method for controlling undesired plant growth, where the compound of formula (I) or an agrochemically acceptable salt thereof according to claim 1 is applied to the plant to be controlled, plant parts, plant seeds or an area on which undesired plant growth takes place.

13. Method according to claim 12, where the undesired plant growth is selected from grasslike monocotyledonous weeds.

14. Method according to claim 12, where the plant growth of resistant grasses in useful plants is controlled, and where the herbicidal composition is applied to the weed to be controlled.

15. Method according to claim 14, where the useful plant is selected from wheat, barley, rye, oats, rice, sugar cane, soybean, rapeseed, sunflower and corn.

16. A compound of formula (I) or an agrochemically acceptable salt thereof according to claim 1 for controlling harmful plants.

17. A compound according to claim 16, wherein the compound of the formula (I) or an agrochemically acceptable salt thereof is used for controlling one or more harmful plants in a crop of useful plants.

18. A compound according to claim 16, wherein the useful plants are transgenic useful plants.
